(19) United States
(12) Patent Application Publication
(10) Pub. No.: US 2025/0057848 A1
(43) Pub. Date: Feb. 20, 2025

(54) GAL475 COMPOSITIONS AND METHODS OF USE THEREOF

(71) Applicant: NEURIM PHARMACEUTICALS (1991) LTD., Tel Aviv (IL)

(72) Inventors: Ran FURMAN, Tel Aviv (IL); Nava ZISAPEL, Tel Aviv (IL)

(73) Assignee: NEURIM PHARMACEUTICALS (1991) LTD., Tel Aviv (IL)

(21) Appl. No.: 18/802,285

(22) Filed: Aug. 13, 2024

Related U.S. Application Data
(60) Provisional application No. 63/519,310, filed on Aug. 14, 2023.

(51) Int. Cl. A61K 31/519; A61P 3/04

(52) U.S. Cl. CPC A61K 31/519; A61P 3/04

(57) ABSTRACT

Methods of treating a subject suffering from a metabolic syndrome or disease, diabetes, obesity, overweight, a food intake disorder, hyperphagia, hyperlipidemia, insulin resistance or a method of lowering body weight in a subject are provided. The methods involve administering a composition comprising a compound of Formula I including, for example, GAL475 (1-(2,6-Bis-methylamino-8-propylamino-pyrimido[5,4-d]pyrimidin-4-yl amino)-2-methyl-propan-2-ol), or a pharmaceutically acceptable salt, solvate, hydrate, enantiomer, stereoisomer, tautomer, or isotopic variant thereof, and a pharmaceutically acceptable carrier or excipient, to the subject.

### CROSS-REFERENCE TO RELATED APPLICATION

This application claims priority benefit and priority to U.S. Appl. No. 63/519,310, filed Aug. 14, 2023, the disclosure of which is incorporated herein in its entirety.

### FIELD OF THE DISCLOSURE

The present disclosure generally relates to compounds including GAL475 (1-(2,6-Bis-methylamino-8-propylamino-pyrimido[5,4-d]pyrimidin-4-yl amino)-2-methyl-propan-2-ol) molecules or a salt, solvate, enantiomer, polymorph, crystal, diastereoisomer, or tautomer thereof in a composition or in the manufacture of a medicament for use in methods of treating diseases or conditions including metabolic syndrome, an overweight condition, diabetes, a food intake disorder, hyperphagia, hyperlipidemia, insulin resistance, obesity, and associated comorbidities.

### BACKGROUND

Metabolic syndromes may result from a cluster of components that reflect overnutrition, sedentary lifestyles, and excess adiposity, and include conditions like obesity and diabetes mellitus. These conditions can lead to various unhealthy conditions including increased blood pressure, high blood sugar, excess body fat around the waist and organs, abnormal cholesterol or triglyceride levels, and comorbidities like prothrombotic states, inflammatory conditions, fatty liver disease, and reproductive disorders. The prevalence of metabolic syndrome is increasing to epidemic proportions not only in the United States and the remainder of the urbanized world, but also in developing nations.

The main underlying causes resulting in metabolic syndrome are abdominal adiposity and insulin resistance. Diabetes mellitus is characterized by hyperglycemia in one of two forms: a non-insulin-dependent or maturity onset form, also known as Type 2 diabetes and an insulin-dependent or juvenile onset form, also known as Type 1 diabetes. The manifestations of clinical symptoms of Type 2 diabetes and the underlying obesity usually appear at an age over 40. In contrast, Type 1 diabetes usually shows a rapid onset of the disease often before 30. The disease is a metabolic disorder in humans with a prevalence of approximately one percent in the general population, with a quarter being Type 1 and the rest being Type 2 diabetes. Subsequently, the treatment of diabetes mellitus is determined by its etiopathology and most commonly includes insulin administration and lifestyle changes (diet, exercise, etc.).

Chronic hyperglycemia in diabetes is associated with end organ damage, dysfunction, and failure, including the retina, kidney, nervous system, heart, and blood vessels (Uazman Alam et al., Handbook of Clinical Neurology Volume 126, 2014).

Obesity is a chronic disease that is highly prevalent in modern society and is associated with numerous medical problems including insulin resistance, hypertension, hypercholesterolemia, coronary heart disease, cancer, and sleep-breathing disorders. Obesity is now so common within the world's population that it is beginning to replace undernutrition and infectious diseases as the most significant contributor to ill health and morbidity. As of 2021, approximately 650 million adults and 340 million children and adolescents suffer from obesity and it is the fourth leading risk for global deaths in the world (Tchang B G et al., Endotext. South Dartmouth: MDtext.com; 2021; Sorensen TIA et al., Handb Exp Pharmacol. 2022; 274:3-27). The World Health Organization (WHO) defines overweight as a body mass index (BMI) of 25-29.9 kg/m2 and obesity as a BMI>30 kg/m2, but this does not take into account the morbidity and mortality associated with more modest degrees of an overweight condition, nor the detrimental effect of intra-abdominal fat. The global epidemic of obesity results from a combination of genetic susceptibility, increased availability of high-energy foods and decreased requirement for physical activity in modern society. Obesity should no longer be regarded simply as a cosmetic problem affecting certain individuals, but an epidemic that threatens global well-being (Peter G. Kopelman Nature 404, 2000).

The cornerstone of obesity treatment involves sustainable lifestyle changes, including a healthy diet combined with physical exercise. Other treatments include surgeries (e.g., gastric sleeve procedure) and medications. The FDA approved several drugs for short term use in obesity (e.g., Phentermine and Diethylpropion). These drugs work by increasing the secretion of norepinephrine and, to a lesser extent, dopamine in neurons and consequently decreasing appetite. Their side effects include impotency, anxiety, nausea, and cardiovascular side effects. The FDA also approved several drugs for long term use in obesity, including orlistat (a lipase inhibitor, which reduces the absorption of fatty acids in the blood), phentermine-topiramate (an amphetamine-related appetite suppressor, which acts likely by inducing the release of adrenalin and suppression of appetite), bupropion-naltrexone (an appetite suppressor, which similarly to the short term drugs, reduces norepinephrine and dopamine reuptake) and liraglutide, setmelanotide, and semaglutide (injectable peptides that work as Glucagon-Like Peptide-1 (GLP-1) receptors agonists).

GLP-1 is a hormone that is released in the gastrointestinal tract in response to eating. One role of GLP-1 is to prompt the body to potentiate glucose-dependent insulin secretion from the pancreatic beta-cells which subsequently reduces blood glucose (sugar). In addition, GLP-1 suppresses glucagon secretion by alpha-cells, leading to a glucose-dependent reduction in hepatic glucose production (Reshma Ramracheya et al., Physiol Rep. 2018 Sep; 6(17)). Glucagon may be regulated in a paracrine manner, by the secretion of somatostatin from the neighboring delta-cell. GLP-1 receptor agonists have been used for the treatment of type 2 diabetes since they mimic the actions of native GLP-1 on pancreatic islet cells, stimulating insulin release, while inhibiting glucagon release, in a glucose-dependent manner. GLP-1 in higher amounts also interacts with the parts of the brain that reduce appetite and signal a feeling of fullness. The observation of weight loss has led to exploration of their potential as anti-obesity agents, with liraglutide 3.0 mg day-1 approved for weight management in the US on Dec. 23, 2014, and in the EU on Mar. 23, 2015 (Donna Ryan and Andres Acosta. Obesity (Silver Spring). 2015 June; 23(6)). Currently, two specific GLP-1 agonists are approved for the treatment of diabetes and obesity (liraglutide and semaglutide) while other agonists are often being used off-label as a treatment for obesity. Several studies showed that GLP-1 agonists reduce cardiovascular events and mortality and protect chondrocytes against endoplasmic reticulum stress, apoptosis, and inflammation via decreased release of inflammatory mediators (Wei Peng et al., Aging Dis. 2022 April; 13(2)). Other ways GLP-1 agonists work include, improving beta cell function through gene regulation, inhibiting glucagon production, and delaying gastric emptying. (Peyton W. Moore Adv Ther. 2023 March;40(3)). One of the main issues when using GLP-1 agonists for obesity treatment is the side effects, which include gastrointestinal issues, nausea, diarrhea, constipation, dyspepsia, abdominal pain, and vomiting; this may require a slow titration in order for patients to better tolerate the medication. In addition, since these drugs are peptides, they can only be introduced via intraparietal injection and cannot be taken orally.

There are currently three FDA-approved semaglutide products: Ozempic® injection, Rybelsus® tablets, and Wegovy® injection. Ozempic and Rybelsus are approved for lowering blood sugar levels in adults with type 2 diabetes mellitus, in addition to diet and exercise. Ozempic® is also approved for reducing the risk of heart attack, stroke, or death in adults with type 2 diabetes mellitus and known heart disease. Wegovy® injection is approved for helping adults and children aged 12 years and older with obesity or some adults with excess weight (overweight), who also have weight-related medical problems, to lose weight and keep the weight off, in addition to diet and exercise.

There is an unmet need for new treatments for metabolic syndromes, diabetes, obesity, and for weight loss that avoid the side effects of existing therapies while offering a more tolerable route of administration.

### SUMMARY OF THE INVENTION

In one aspect, the present disclosure includes a method of treating a subject suffering from a metabolic syndrome or disease comprising administering a composition comprising GAL475 (1-(2,6-Bis-methylamino-8-propylamino-pyrimido[5,4-d]pyrimidin-4-yl amino)-2-methyl-propan-2-ol), or a pharmaceutically acceptable salt, solvate, hydrate, enantiomer, stereoisomer, tautomer, or isotopic variant thereof, and a pharmaceutically acceptable carrier or excipient, to the subject.

The invention provides a compound of formula (I), or pharmaceutically acceptable salt, solvate, hydrate, enantiomer, stereoisomer, tautomer, or isotopic variant thereof:

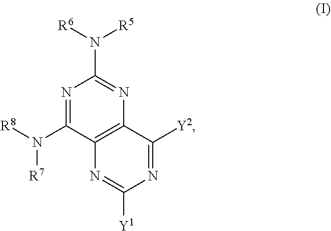

wherein in (I): one of the substituents selected from the group consisting of Y¹ and Y² is selected from the group consisting of —N(R¹)—L—C(R⁹)(R¹⁰)OH,

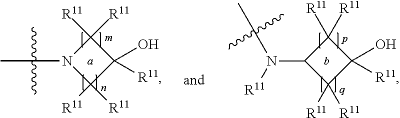

and the other substituent is —N(R¹)R²;
R¹, R⁸ and R⁷ are independently selected from the group consisting of hydrogen and optionally substituted C₁-C₃ alkyl;
R² is selected from the group consisting of alkyl, cycloalkyl, alkenyl, alkynyl, phenyl, phenylalkyl, aryl, arylalkyl, heteroarylalkyl and heteroaryl, wherein the alkyl, cycloalkyl, alkenyl, alkynyl, phenyl, phenylalkyl, aryl, arylalkyl, heteroarylalkyl or heteroaryl group is independently optionally substituted;
R⁶ and R⁸ are independently selected from the group consisting of alkyl, cycloalkyl, alkenyl, alkynyl, phenyl, phenylalkyl, aryl, arylalkyl, heteroarylalkyl and heteroaryl,
wherein the alkyl, cycloalkyl, alkenyl, alkynyl, phenyl, phenylalkyl, aryl, arylalkyl, heteroarylalkyl or heteroaryl group is independently optionally substituted;
R⁹ and R¹⁰ are independently selected from the group consisting of H and optionally substituted C₁-C₃-alkyl; or R⁹ and R¹⁰ combine with the carbon atom to which they are bound so as to form an optionally substituted C₃-C₆ cycloalkyl group;
each instance of R¹¹ is independently selected from the group consisting of H and optionally substituted C₁-C₃-alkyl; wherein a —C(R¹¹)₂—C(R¹¹)₂— group within ring b is optionally replaced by an optionally substituted 1,2-phenylene group that is fused with ring b;
each occurrence of independently optionally substituted C₁-C₃ alkylene;
m and n are independently selected from the group consisting of 1, 2, 3 and 4, such that 2≤(m+n)≤4;
p and q are independently elected from the group consisting of 0, 1, 2, 3 and 4, such that 2≤(p+q)≤4;
with the proviso that the alkyl group is not substituted with a hydroxy group.

In certain embodiments, the compound of formula (I) may include compound of formula (IIa), or a pharmaceutically acceptable salt, solvate, hydrate, enantiomer,

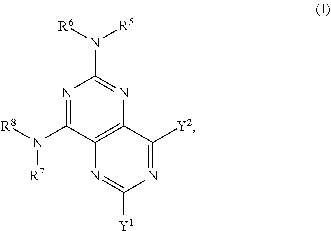

stereoisomer, tautomer, or isotopic variant thereof: (I), wherein in (I): one of the substituents selected from the group consisting of Y¹ and Y² is selected from the group consisting of —N(R¹¹)—L—C(R⁹)(R¹⁰)OH,

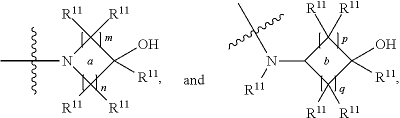

and the other substituent is —N(R¹)R²;
R¹, R⁵ and R⁷ are independently selected from the group consisting of hydrogen and optionally substituted C₁-C₃ alkyl;
R² is selected from the group consisting of alkyl, cycloalkyl, alkenyl, alkynyl, phenyl, phenylalkyl, aryl, arylalkyl, heteroarylalkyl and heteroaryl, wherein the alkyl, cycloalkyl, alkenyl, alkynyl, phenyl, phenylalkyl, aryl, arylalkyl, heteroarylalkyl or heteroaryl group is independently optionally substituted;
R⁶ and R⁸ are independently selected from the group consisting of alkyl, cycloalkyl, alkenyl, alkynyl, phenyl, phenylalkyl, aryl, arylalkyl, heteroarylalkyl and heteroaryl, wherein the alkyl, cycloalkyl, alkenyl, alkynyl, phenyl, phenylalkyl, aryl, arylalkyl, heteroarylalkyl or heteroaryl group is independently optionally substituted;
R⁹ and R¹⁰ are independently selected from the group consisting of H and optionally substituted C₁-C₃-alkyl; or R⁹ and R¹⁰ combine with the carbon atom to which they are bound so as to form an optionally substituted C₃-C₆ cycloalkyl group;
each instance of R¹¹ is independently selected from the group consisting of H and optionally substituted C₁-C₃-alkyl; wherein a —C(R¹¹)₂—C(R¹¹)₂— group within ring b is optionally replaced by an optionally substituted 1,2-phenylene group that is fused with ring b;
each occurrence of independently optionally substituted C₁-C₃ alkylene;
m and n are independently selected from the group consisting of 1, 2, 3 and 4, such that 2≤(m+n)≤4;
p and q are independently elected from the group consisting of 0, 1, 2, 3 and 4, such that 2≤(p+q)≤4;
with the proviso that the alkyl group is not substituted with a hydroxy group.

In certain embodiments, the compound of formula (I) is the compound of formula (IIb):

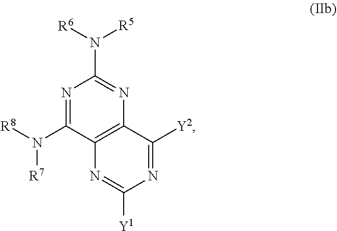

wherein in (IIb): one of the substituents selected from the group consisting of Y¹ and Y² is

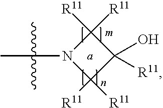

and the other substituent is —N(R¹)R²; R¹, R⁵ and R⁷ are independently selected from the group consisting of H and optionally substituted C₁-C₃ alkyl; R² is selected from the group consisting of alkyl, cycloalkyl, alkenyl, alkynyl, phenyl, phenylalkyl, aryl, arylalkyl, heteroarylalkyl and heteroaryl, wherein the alkyl, cycloalkyl, alkenyl, alkynyl, phenyl, phenylalkyl, aryl, arylalkyl, heteroarylalkyl or heteroaryl group is independently optionally substituted; L is optionally substituted C₁-C₃ alkylene; and R⁶ and R⁸ are independently selected from the group consisting of alkyl, cycloalkyl, alkenyl, alkynyl, phenyl, phenylalkyl, aryl, arylalkyl, heteroarylalkyl and heteroaryl, wherein the alkyl, cycloalkyl, alkenyl, alkynyl, phenyl, phenylalkyl, aryl, arylalkyl, heteroarylalkyl or heteroaryl group is independently optionally substituted; each instance of R¹¹ is independently selected from the group consisting of hydrogen and optionally substituted C₁-C₃-alkyl; L is optionally substituted C₁-C₃ alkylene; m and n are independently selected from the group consisting of 1, 2, 3 and 4, such that 2≤m+n≤4; with the proviso that the alkyl group is not substituted with a hydroxy group.

In certain embodiments, the compound of formula (I) is a compound of formula (IIc):

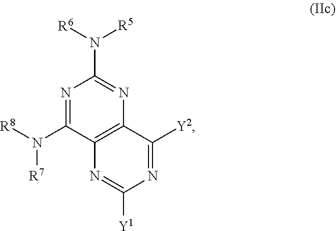

wherein in (IIc): one of the substituents selected from the group consisting of Y¹ and Y² is

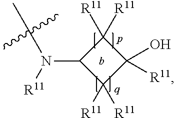

and the other substituent is —N(R¹¹)R²; R¹, R⁵ and R⁷ are independently selected from the group consisting of hydrogen and optionally substituted C₁-C₃ alkyl; R² is selected from the group consisting of alkyl, cycloalkyl, alkenyl, alkynyl, phenyl, phenylalkyl, aryl, arylalkyl, heteroarylalkyl and heteroaryl, wherein the alkyl, cycloalkyl, alkenyl, alkynyl, phenyl, phenylalkyl, aryl, arylalkyl, heteroarylalkyl or heteroaryl group is independently optionally substituted; R⁶ and R⁸ are independently selected from the group consisting of alkyl, cycloalkyl, alkenyl, alkynyl, phenyl, phenylalkyl, aryl, arylalkyl, heteroarylalkyl and heteroaryl, wherein the alkyl, cycloalkyl, alkenyl, alkynyl, phenyl, phenylalkyl, aryl, arylalkyl, heteroarylalkyl or heteroaryl group is independently optionally substituted; R⁹ and R¹⁰ are independently selected from the group consisting of H and optionally substituted C₁-C₃-alkyl; or R⁹ and R¹⁰ combine with the carbon atom to which they are bound so as to form an optionally substituted C₃-C₆ cycloalkyl group; each instance of R¹¹ is independently selected from the group consisting of H and optionally substituted C₁-C₃-alkyl; wherein a —C(R¹¹)₂—C(R¹¹)₂— group within ring b is optionally replaced by an optionally substituted 1,2-phenylene group that is fused with ring b; each occurrence of L is independently optionally substituted C₁-C₃ alkylene; p and q are s independently elected from the group consisting of 0, 1, 2, 3 and 4, such that 2<p+q≤4; with the proviso that the alkyl group is not substituted with a hydroxy group.

In certain embodiments of compounds of formulas (I), (IIa), (IIb) and (IIc), each occurrence of the alkyl group is optionally substituted with one or more substituents independently selected from the group consisting of C₁-C₆ alkyl, F, Cl, Br, I, and CN; each occurrence of the cycloalkyl, alkenyl or alkynyl group is optionally substituted with one or more substituents independently selected from the group consisting of C₁-C₆ alkyl, F, Cl, Br, I, and CN; each occurrence of the phenyl, phenylalkyl, aryl, arylalkyl, heteroarylalkyl or heteroaryl group is optionally substituted with one or more substituents independently selected from the group consisting of C₁-C₆ alkyl, C₁-C₆ alkoxy, hydroxy, F, Cl, Br, I, nitro, —NH₂, —NH(C₁-C₆ alkyl), —N(C₁-C₆ alkyl)(C₁-C₆ alkyl), —S(═O)₀₋₂(C₁-C₆ alkyl), —C(═O)OH and —C(═O)OC₁-C₆ alkyl.

In certain embodiments of compounds of formula (I), R¹, R⁵ and R⁷ are H. In certain embodiments of compounds of formula (I), R¹, R⁵ and R⁷ are independently optionally substituted C₁-C₃ alkyl. In certain embodiments of compounds of formula (I), R¹, R⁵ and R⁷ are H; and R⁹ and R¹⁰ are H. In certain embodiments of compounds of formula (I), R¹, R⁵ and R⁷ are H; R⁹ is H; and R¹⁰ are CH₃.

In certain embodiments, a compound of formula (I) is selected from the group consisting of. 2-(2,6-Bis-methylamino-8-propylamino-pyrimido[5,4-d]pyrimidin-4-ylamino)-ethanol (4); 2-[(2,6-Bis-methylamino-8-propylamino-pyrimido[5,4-d]pyrimidin-4-yl)-methyl-amino]-ethanol (6); 3-(2,6-Bis-methylamino-8-propylamino-pyrimido[5,4-d]pyrimidin-4-ylamino)-propan-1-ol (8); 1-(2,6-Bis-methylamino-8-propylamino-pyrimido[5,4-d]-pyrimidin-4-ylamino)-propan-2-ol (10); (S)-1-(2,6-Bis-methylamino-8-propylamino-pyrimido[5,4-d]pyrimidin-4-ylamino)-propan-2-ol (12); (R)-1-(2,6-Bis-methylamino-8-propylamino-pyrimido[5,4-d]pyrimidin-4-ylamino)-propan-2-ol (14); 2-(2,6-Bis-methylamino-8-propylamino-pyrimido[5,4-d]-pyrimidin-4-ylamino)-2-methyl-propan-1-ol (16); (S)-2-(2,6-Bis-methylamino-8-propylamino-pyrimido[5,4-d]-pyrimidin-4-ylamino)-propan-1-ol (18); (R)-2-(2,6-Bis-methylamino-8-propylamino-pyrimido[5,4-d]-pyrimidin-4-ylamino)-propan-1-ol (20); 3-(2,6-Bis-methylamino-8-propylamino-pyrimido[5,4-d]pyrimidin-4-ylamino)-1,1,1-trifluoro-propan-2-ol (22); 1-(2,6-Bis-methylamino-8-propylamino-pyrimido[5,4-d]pyrimidin-4-ylamino)-butan-2-ol (24); 3-(2,6-Bis-methylamino-8-propylamino-pyrimido[5,4-d]pyrimidin-4-ylamino)-butan-2-ol (26); 2-(2,6-Bis-ethylamino-8-propylamino-pyrimido[5,4-d]pyrimidin-4-ylamino)-ethanol (27); 2-[8-Propylamino-2,6-bis-(2,2,2-trifluoro-ethylamino)-pyrimido[5,4-d]pyrimidin-4-ylamino]-ethanol (28); 1-(2,6-Bis-methylamino-8-propylamino-pyrimido[5,4-d]pyrimidin-4-ylamino)-2-methyl-propan-2-ol (31); 1-(2,6-Bis-ethylamino-8-propylamino-pyrimido[5,4-d]-pyrimidin-4-ylamino)-2-methyl-propan-2-ol (32); 1-[2,6-Bis-(2,2-difluoro-ethylamino)-8-propylamino-pyrimido[5,4-d]-pyrimidin-4-ylamino]-2-methyl-propan-2-ol (33); 2-Methyl-1-[8-propylamino-2,6-bis-(2,2,2-trifluoro-ethylamino)-pyrimido[5,4-d]pyrimidin-4-ylamino]-propan-2-ol (34); 1-[8-(2,2-difluoro-ethylamino)-2,6-bis-methylamino-pyrimido[5,4-d]pyrimidin-4-ylamino]-2-methyl-propan-2-ol (36); 1-{2,6-bis-methylamino-8-[(pyrimidin-2-ylmethyl)-amino]-pyrimido[5,4-d]pyrimidin-4-ylamino}-2-methyl-propan-2-ol (38); 1-[8-((R)-sec-butylamino)-2,6-bis-methylamino-pyrimido[5,4-d]-pyrimidin-4-ylamino]-2-methyl-propan-2-ol (40); 1-[8-((S)-sec-butylamino)-2,6-bis-methylamino-pyrimido[5,4-d]-pyrimidin-4-ylamino]-2-methyl-propan-2-ol (42); 1-(8-benzylamino-2,6-bis-methylamino-pyrimido[5,4-d]pyrimidin-4-ylamino)-2-methyl-propan-2-ol (44); 1-[8-(cyclopropylmethyl-amino)-2,6-bis-methylamino-pyrimido[5,4-d]pyrimidin-4-ylamino]-2-methyl-propan-2-ol (46); 1-[8-(2,2-difluoro-ethylamino)-2,6-bis-ethylamino-pyrimido[5,4-d]pyrimidin-4-ylamino]-2-methyl-propan-2-ol (47); 2-methyl-1-(2,6,8-tris-methylamino-pyrimido[5,4-d]-pyrimidin-4-ylamino)-propan-2-ol (48); 2-methyl-1-(2,6,8-tris-ethylamino-pyrimido[5,4-d]-pyrimidin-4-ylamino)-propan-2-ol (49); 2-(2,6,8-tris-methylamino-pyrimido[5,4-d]pyrimidin-4-ylamino)-ethanol (52); 2-[8-(cyclopropylmethyl-amino)-2,6-bis-methylamino-pyrimido[5,4-d]pyrimidin-4-ylamino]-ethanol (54); 2-[8-(2-methoxy-ethylamino)-2,6-bis-methylamino-pyrimido[5,4-d]pyrimidin-4-ylamino]-ethanol (56); 2-(2,6-bis-methylamino-8-prop-2-ynylamino-pyrimido[5,4-d]pyrimidin-4-ylamino)-ethanol (58); 2-[8-(2,2-difluoro-ethylamino)-2,6-bis-methylamino-pyrimido[5,4-d]-pyrimidin-4-ylamino]-ethanol (60); 2-[2,6-bis-methylamino-8-(2,2,2-trifluoro-ethylamino)-pyrimido[5,4-d]pyrimidin-4-ylamino]-ethanol (62); 2-(8-benzylamino-2,6-bis-methylamino-pyrimido[5,4-d]-pyrimidin-4-ylamino)-ethanol (64); 3-(8-ethylamino-2,6-bis-methylamino-pyrimido[5,4-d]-pyrimidin-4-ylamino)-propan-1-ol (67); 1-(2,6-bis-methylamino-8-propylamino-pyrimido[5,4-d]pyrimidin-4-yl)-pyrrolidin-3-ol (71); 1-[(2,6-bis-methylamino-8-propylamino-pyrimido[5,4-d]pyrimidin-4-ylamino)-methyl]-cyclobutanol (72); 1-[(2,6-bis-methylamino-8-propylamino-pyrimido[5,4-d]-pyrimidin-4-yl)-methyl-amino]-propan-2-ol (73); 3-[(2,6-bis-methylamino-8-propylamino-pyrimido[5,4-d]pyrimidin-4-ylamino)-methyl]-pentan-3-ol (74); 1-[(2,6-bis-methylamino-8-propylamino-pyrimido[5,4-d]pyrimidin-4-yl)-methyl-amino]-2-methyl-propan-2-ol (76); (1R,2S)-1-(2,6-bis-methylamino-8-propylamino-pyrimido[5,4-d]-pyrimidin-4-ylamino)-indan-2-ol (77); (1S,2S)-1-(2,6-bis-methylamino-8-propylamino-pyrimido[5,4-d]-pyrimidin-4-ylamino)-indan-2-ol (78); (1S,2R)-1-(2,6-bis-methylamino-8-propylamino-pyrimido[5,4-d]pyrimidin-4-ylamino)-indan-2-ol (79); (1R,2R)-1-(2,6-bis-methylamino-8-propylamino-pyrimido[5,4-d]-pyrimidin-4-ylamino)-indan-2-ol (80); (2-(2,6-bis-methylamino-8-propylamino-pyrimido[5,4-d]-pyrimidin-4-ylamino)-indan-1-ol (81); (1R,2S)-2-(2,6-bis-methylamino-8-propylamino-pyrimido[5,4-d]pyrimidin-4-ylamino)-cyclohexanol (82); (1S,2S)-2-(2,6-bis-methylamino-8-propylamino-pyrimido[5,4-d]pyrimidin-4-ylamino)-cyclohexanol (83); (1S,2R)-2-(2,6-bis-methylamino-8-propylamino-pyrimido[5,4-d]pyrimidin-4-ylamino)-cyclohexanol (84); (1R,2R)-2-(2,6-bis-methylamino-8-propylamino-pyrimido[5,4-d]pyrimidin-4-ylamino)-cyclohexanol (85); (1S,2S)-2-(2,6-bis-methylamino-8-propylamino-pyrimido[5,4-d]pyrimidin-4-ylamino)-cyclopentanol (86); (1R,2R)-2-(2,6-bis-methylamino-8-propylamino-pyrimido[5,4-d]pyrimidin-4-ylamino)-cyclopentanol (87); 2-[6-(cyclopropylmethyl-amino)-4,8-bis-methylamino-pyrimido[5,4-d]pyrimidin-2-ylamino]-ethanol (90); 2-(4,8-bis-methylamino-6-propylamino-pyrimido[5,4-d]pyrimidin-2-ylamino)-ethanol (91); 2-(6-dimethylamino-4,8-bis-methylamino-pyrimido[5,4-d]-pyrimidin-2-ylamino)-ethanol (92); 1-(4,8-bis-methylamino-6-propylamino-pyrimido[5,4-d]pyrimidin-2-ylamino)-2-methyl-propan-2-ol (94); 1-(4,8-bis-methylamino-6-propylamino-pyrimido[5,4-d]-pyrimidin-2-ylamino)-propan-2-ol (95); 1-[(4,8-bis-methylamino-6-propylamino-pyrimido[5,4-d]pyrimidin-2-yl)-methyl-amino]-2-methyl-propan-2-ol (96); 1-[(4,8-bis-methylamino-6-propylamino-pyrimido[5,4-d]pyrimidin-2-yl)-methyl-amino]-propan-2-ol (97); 1-[6-((R)-sec-butylamino)-4,8-bis-methylamino-pyrimido[5,4-d]-pyrimidin-2-ylamino]-2-methyl-propan-2-ol (99); (R)-1-[6-((R)-sec-butylamino)-4,8-bis-methylamino-pyrimido[5,4-d]pyrimidin-2-ylamino]-propan-2-ol (100); (S)-1-[6-((R)-sec-butylamino)-4,8-bis-methylamino-pyrimido[5,4-d]pyrimidin-2-ylamino]-propan-2-ol (101); 1-[6-((S)-sec-butylamino)-4,8-bis-methylamino-pyrimido[5,4-d]-pyrimidin-2-ylamino]-2-methyl-propan-2-ol (103); (R)-1-[6-((S)-sec-butylamino)-4,8-bis-methylamino-pyrimido[5,4-d]pyrimidin-2-ylamino]-propan-2-ol (104); (S)-1-[6-((S)-sec-butylamino)-4,8-bis-methylamino-pyrimido[5,4-d]pyrimidin-2-ylamino]-propan-2-ol (105); 1-[6-(2,2-difluoro-ethylamino)-4,8-bis-methylamino-pyrimido[5,4-d]pyrimidin-2-ylamino]-2-methyl-propan-2-ol (107); 1-{4,8-bis-methylamino-6-[(pyrimidin-2-ylmethyl)-amino]-pyrimido[5,4-d]pyrimidin-2-ylamino}-2-methyl-propan-2-ol (109); 3-(4,8-bis-methylamino-6-propylamino-pyrimido[5,4-d]pyrimidin-2-ylamino)-1,1,1-trifluoro-propan-2-ol (111); (S)-1-(4,8-bis-methylamino-6-propylamino-pyrimido[5,4-d]pyrimidin-2-ylamino)-propan-2-ol (113); (R)-1-(4,8-bis-methylamino-6-propylamino-pyrimido[5,4-d]pyrimidin-2-ylamino)-propan-2-ol (115); 1-(4,8-bis-methylamino-6-propylamino-pyrimido[5,4-d]pyrimidin-2-ylamino)-butan-2-ol (117); 3-(4,8-Bis-methylamino-6-propylamino-pyrimido[5,4-d]pyrimidin-2-ylamino)-butan-2-ol (119); (1R,2S)-1-(4,8-bis-methylamino-6-propylamino-pyrimido[5,4-d]-pyrimidin-2-ylamino)-indan-2-ol (123); (1S,2S)-1-(4,8-bis-methylamino-6-propylamino-pyrimido[5,4-d]-pyrimidin-2-ylamino)-indan-2-ol (125); (1S,2R)-1-(4,8-bis-methylamino-6-propylamino-pyrimido[5,4-d]-pyrimidin-2-ylamino)-indan-2-ol (127); (1R,2R)-1-(4,8-bis-methylamino-6-propylamino-pyrimido[5,4-d]-pyrimidin-2-ylamino)-indan-2-ol (129); (1R,2S)-2-(4,8-bis-methylamino-6-propylamino-pyrimido[5,4-d]pyrimidin-2-ylamino)-cyclohexanol (131); (1S,2S)-2-(4,8-bis-methylamino-6-propylamino-pyrimido[5,4-d]pyrimidin-2-ylamino)-cyclohexanol (133); (1S,2R)-2-(4,8-bis-methylamino-6-propylamino-pyrimido[5,4-d]pyrimidin-2-ylamino)-cyclohexanol (135); (1R,2R)-2-(4,8-bis-methylamino-6-propylamino-pyrimido[5,4-d]pyrimidin-2-ylamino)-cyclohexanol (137); (1S,2S)-2-(4,8-bis-methylamino-6-propylamino-pyrimido[5,4-d]pyrimidin-2-ylamino)-cyclopentanol (139); (1R,2R)-2-(4,8-bis-methylamino-6-propylamino-pyrimido[5,4-d]pyrimidin-2-ylamino)-cyclopentanol (141); (S)-1-[6-(cyclopropylmethyl-amino)-4,8-bis-methylamino-pyrimido[5,4-d]pyrimidin-2-ylamino]-propan-2-ol (142); (S)-1-(6-allylamino-4,8-bis-methylamino-pyrimido[5,4-d]pyrimidin-2-ylamino)-propan-2-ol (143); (R)-1-[6-(cyclopropylmethyl-amino)-4,8-bis-methylamino-pyrimido[5,4-d]pyrimidin-2-ylamino]-propan-2-ol (144); (R)-1-(6-allylamino-4,8-bis-methylamino-pyrimido[5,4-d]pyrimidin-2-ylamino)-propan-2-ol (145); 1-[6-(cyclopropylmethyl-amino)-4,8-bis-methylamino-pyrimido[5,4-d]pyrimidin-2-ylamino]-butan-2-ol (146); 1-(6-ethylamino-4,8-bis-methylamino-pyrimido[5,4-d]pyrimidin-2-ylamino)-butan-2-ol (147); 2-methyl-1-(4,6,8-tris-methylamino-pyrimido[5,4-d]pyrimidin-2-ylamino)-propan-2-ol (149); 2-(6-allylamino-4,8-bis-methylamino-pyrimido[5,4-d]pyrimidin-2-ylamino)-ethanol (154); (S)-1-[(6-allylamino-4,8-bis-methylamino-pyrimido[5,4-d]-pyrimidin-2-yl)-propyl-amino]-propan-2-ol (155); (S)-1-[(6-allylamino-4,8-bis-methylamino-pyrimido[5,4-d]pyrimidin-2-yl)-methyl-amino]-propan-2-ol (156); (R)-1-[6-(2-methyl-allylamino)-4,8-bis-methylamino-pyrimido[5,4-d]-pyrimidin-2-ylamino]-propan-2-ol (158); (S)-1-[6-(2-methyl-allylamino)-4,8-bis-methylamino-pyrimido[5,4-d]-pyrimidin-2-ylamino]-propan-2-ol (159); 2-(4,8-bis-ethylamino-6-propylamino-pyrimido[5,4-d]pyrimidin-2-ylamino)-ethanol (162); 1-(4,8-bis-ethylamino-6-propylamino-pyrimido[5,4-d]pyrimidin-2-ylamino)-2-methyl-propan-2-ol (163); (S)-1-(4,6,8-Tris-ethylamino-pyrimido[5,4-d]pyrimidin-2-ylamino)-propan-2-ol (165); (S)-1-(4,8-bis-ethylamino-6-propylamino-pyrimido[5,4-d]-pyrimidin-2-ylamino)-propan-2-ol (166); (R)-1-(4,6,8-tris-ethylamino-pyrimido[5,4-d]pyrimidin-2-ylamino)-propan-2-ol (168); (R)-1-(4,8-bis-ethylamino-6-propylamino-pyrimido[5,4-d]-pyrimidin-2-ylamino)-propan-2-ol (169); (R)-1-[4,8-bis-ethylamino-6-(2-methyl-allylamino)-pyrimido[5,4-d]-pyrimidin-2-ylamino]-propan-2-ol (174); (S)-1-[4,8-bis-ethylamino-6-(2-methyl-allylamino)-pyrimido[5,4-d]-pyrimidin-2-ylamino]-propan-2-ol (175); pharmaceutically acceptable salt, solvate, hydrate, enantiomer, stereoisomer, tautomer, isotopic variant, and any combinations thereof.

A detailed description of the compounds is disclosed in U.S. patent number U.S. Ser. No. 10/294,228B2, which is incorporated herein by reference.

In one aspect, the present disclosure includes a method of lowering body weight in a subject in need thereof, the method comprising administering a composition comprising a compound of formula (I) or a pharmaceutically acceptable salt, solvate, hydrate, enantiomer, stereoisomer, tautomer, or isotopic variant thereof, and in particular aspects GAL475 (1-(2,6-Bis-methylamino-8-propylamino-pyrimido[5,4-d]pyrimidin-4-yl amino)-2-methyl-propan-2-ol), or a pharmaceutically acceptable salt, solvate, hydrate, enantiomer, stereoisomer, tautomer, or isotopic variant thereof, and a pharmaceutically acceptable carrier or excipient, to the subject.

In one aspect, the present disclosure includes a method of treating a subject having diabetes or at risk of developing diabetes, the method comprising administering a composition comprising a compound of formula (I) or a pharmaceutically acceptable salt, solvate, hydrate, enantiomer, stereoisomer, tautomer, or isotopic variant thereof, and in particular aspects GAL475 (1-(2,6-Bis-methylamino-8-propylamino-pyrimido[5,4-d]pyrimidin-4-yl amino)-2-methyl-propan-2-ol), or a pharmaceutically acceptable salt, solvate, hydrate, enantiomer, stereoisomer, tautomer, or isotopic variant thereof, and a pharmaceutically acceptable carrier or excipient, to the subject.

In one aspect, the present disclosure includes a method of treating a subject having obesity, the method comprising administering a composition comprising a compound of formula (I) or a pharmaceutically acceptable salt, solvate, hydrate, enantiomer, stereoisomer, tautomer, or isotopic variant thereof, and in particular aspects GAL475 (1-(2,6-Bis-methylamino-8-propylamino-pyrimido[5,4-d]pyrimidin-4-yl amino)-2-methyl-propan-2-ol), or a pharmaceutically acceptable salt, solvate, hydrate, enantiomer, stereoisomer, tautomer, or isotopic variant thereof, and a pharmaceutically acceptable carrier or excipient, to the subject.

In one aspect, the present disclosure includes a method of treating a subject having a food intake disorder, the method comprising administering a composition comprising a compound of formula (I) or a pharmaceutically acceptable salt, solvate, hydrate, enantiomer, stereoisomer, tautomer, or isotopic variant thereof, and in particular aspects GAL475 (1-(2,6-Bis-methylamino-8-propylamino-pyrimido[5,4-d]pyrimidin-4-yl amino)-2-methyl-propan-2-01), or a pharmaceutically acceptable salt, solvate, hydrate, enantiomer, stereoisomer, tautomer, or isotopic variant thereof, and a pharmaceutically acceptable carrier or excipient, to the subject.

In one aspect, the present disclosure includes a method of treating a subject having hyperphagia, the method comprising administering a composition comprising a compound of formula (I) or a pharmaceutically acceptable salt, solvate, hydrate, enantiomer, stereoisomer, tautomer, or isotopic variant thereof, and in particular aspects GAL475 (1-(2,6-Bis-methylamino-8-propylamino-pyrimido[5,4-d]pyrimidin-4-yl amino)-2-methyl-propan-2-ol), or a pharmaceutically acceptable salt, solvate, hydrate, enantiomer, stereoisomer, tautomer, or isotopic variant thereof, and a pharmaceutically acceptable carrier or excipient, to the subject.

In one aspect, the present disclosure includes a method of treating a subject having hyperlipidemia, the method comprising administering a composition comprising a compound of formula (I) or a pharmaceutically acceptable salt, solvate, hydrate, enantiomer, stereoisomer, tautomer, or isotopic variant thereof, and in particular aspects GAL475 (1-(2,6-Bis-methylamino-8-propylamino-pyrimido[5,4-d]pyrimidin-4-yl amino)-2-methyl-propan-2-01), or a pharmaceutically acceptable salt, solvate, hydrate, enantiomer, stereoisomer, tautomer, or isotopic variant thereof, and a pharmaceutically acceptable carrier or excipient, to the subject.

In one aspect, the present disclosure includes a method of treating a subject having insulin resistance, the method comprising administering a composition comprising a compound of formula (I) or a pharmaceutically acceptable salt, solvate, hydrate, enantiomer, stereoisomer, tautomer, or isotopic variant thereof, and in particular aspects GAL475 (1-(2,6-Bis-methylamino-8-propylamino-pyrimido[5,4-d]pyrimidin-4-yl amino)-2-methyl-propan-2-01), or a pharmaceutically acceptable salt, solvate, hydrate, enantiomer, stereoisomer, tautomer, or isotopic variant thereof, and a pharmaceutically acceptable carrier or excipient, to the subject.

In some aspects, the composition is an immediate release composition.

In some aspects, the composition is a modified-release composition.

In some aspects, the modified release composition is an oral modified-release composition.

In some aspects, the composition comprises an enteric coating.

In some aspects, the composition comprises a gelatin coating.

In some aspects, the composition comprises an excipient, wherein the excipient comprises a binder, disintegrant, diluent, buffer, lubricant, glidant, antioxidant, antimicrobial preservative, colorant, or flavorant.

In some aspects, the compound is coated onto a base particle so as to form a core.

In some aspects, the core is coated with an enteric coating, thereby forming an enterically coated bead.

In some aspects, the dosage form is an oral dosage form.

In some aspects, the oral dosage form is in the form of a capsule, a tablet, or a pharmaceutically acceptable solution.

In some aspects, the composition comprises about 2 mg to about 5000 mg, about 5 mg to about 3000 mg, about 10 mg to about 2000 mg, about 20 mg to about 1500 mg, about 30 mg to about 1000 mg, about 40 mg to about 900 mg, about 50 mg to about 800 mg, about 100 mg to about 700 mg, about 150 mg to about 600 mg, about 200 mg to about 500 mg, about 250 mg to about 400 mg, or about 300 mg to about 350 mg of the compound.

In some aspects, the composition is encapsulated in a capsule.

In some aspects, the capsule contains granules or powders of the compound, or granules or powder comprising a mixture of the compound with the pharmaceutically acceptable carrier or excipient.

In some aspects, the capsule is enterically coated but the granules or powders are not enterically coated.

In some aspects, at least a portion of the granules or powders are enterically coated.

In some aspects, at least a portion of the granules or powders are coated with an enteric coating before encapsulation in the capsule.

In some aspects, a first subportion of the granules or the powders coated with one enteric coating and at least a second subportion of the granules or the coated with a different enteric coating, wherein the first subportion is released in a different region of the intestine of the subject than the second subportion.

In some aspects, the capsule is a liquid-filled capsule further comprising the composition and a pharmaceutically acceptable liquid mixed to form a liquid formulation.

In some aspects, the capsule is enterically coated, and wherein the liquid formulation in the capsule does not comprise an enteric coating.

In some aspects, the method further comprises administering at least one agent known for treating the underlying disease.

In some aspects, the method further comprises at least one additional agent selected from the group consisting of an antidiabetic agent, a weight loss agent, a metabolic syndrome treatment agent, and an anti-obesity agent.

In some aspects, the method further comprises administering the composition or capsule separately from at least one agent.

In some aspects, the method further comprises co-administering the composition or capsule with at least one agent.

In some aspects, the compound and the at least one additional agent are physically mixed in the composition.

In some aspects, the compound and the at least one additional agent are configured in a physically-separated structure in the composition

In some aspects, the subject is a mammal or a bird.

In some aspects, the subject is a human.

In some aspects, the administering is by at least one route selected from the group consisting of nasal, inhalational, topical, oral, buccal, rectal, pleural, peritoneal, vaginal, intramuscular, subcutaneous, transdermal, epidural, intrathecal, and intravenous.

In some aspects, the pharmaceutical acceptable salt comprises an acid addition salt selected from the group consisting of sulfuric, hydrochloric, hydrobromic, hydroiodic, nitric, carbonic, phosphoric, formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, pamoic, methanesulfonic, ethanesulfonic, benzenesulfonic, pantothenic, sulfanilic, stearic, alginic, trifluoromethanesulfonic, 2-hydroxyethanesulfonic, p-tolucncsul ionic, cyclohexylaminosulfonic, β-hydroxybutyric, salicylic, galactaric and galacturonic, or a combination thereof. In some aspects, the salt is (1-(2,6-Bis-methylamino-8-propylamino-pyrimido[5,4-d]pyrimidin-4-yl amino)-2-methyl-propan-2-ol bis hydrochloride).

In one aspect, the present disclosure includes use of a compound of formula (I) or a pharmaceutically acceptable salt, solvate, hydrate, enantiomer, stereoisomer, tautomer, or isotopic variant thereof, and in particular aspects the compound is GAL475 (1-(2,6-Bis-methylamino-8-propylamino-pyrimido[5,4-d]pyrimidin-4-yl amino)-2-methyl-propan-2-ol), and a pharmaceutically acceptable salt, solvate, hydrate, enantiomer, stereoisomer, tautomer, or isotopic variant thereof, in the manufacture of a medicament for inhibiting or treating a metabolic syndrome or disease in a subject in need thereof.

In one aspect, the present disclosure includes use of a compound of formula (I) or a pharmaceutically acceptable salt, solvate, hydrate, enantiomer, stereoisomer, tautomer, or isotopic variant thereof, and in particular aspects the compound is GAL475 (1-(2,6-Bis-methylamino-8-propylamino-pyrimido[5,4-d]pyrimidin-4-yl amino)-2-methyl-propan-2-ol), and a pharmaceutically acceptable salt, solvate, hydrate, enantiomer, stereoisomer, tautomer, or isotopic variant thereof, in the manufacture of a medicament for inducing weight loss in a subject in need thereof.

In one aspect, the present disclosure includes use of a compound of formula (I) or a pharmaceutically acceptable salt, solvate, hydrate, enantiomer, stereoisomer, tautomer, or isotopic variant thereof, and in particular aspects the compound is GAL475 (1-(2,6-Bis-methylamino-8-propylamino-pyrimido[5,4-d]pyrimidin-4-yl amino)-2-methyl-propan-2-ol), and a pharmaceutically acceptable salt, solvate, hydrate, enantiomer, stereoisomer, tautomer, or isotopic variant thereof, in the manufacture of a medicament for treating or preventing diabetes in a subject in need thereof.

In one aspect, the present disclosure includes use of a compound of formula (I) or a pharmaceutically acceptable salt, solvate, hydrate, enantiomer, stereoisomer, tautomer, or isotopic variant thereof, and in particular aspects the compound is GAL475 (1-(2,6-Bis-methylamino-8-propylamino-pyrimido[5,4-d]pyrimidin-4-yl amino)-2-methyl-propan-2-ol), and a pharmaceutically acceptable salt, solvate, hydrate, enantiomer, stereoisomer, tautomer, or isotopic variant thereof, in the manufacture of a medicament for treating a food intake disorder in a subject in need thereof.

In one aspect, the present disclosure includes use of a compound of formula (I) or a pharmaceutically acceptable salt, solvate, hydrate, enantiomer, stereoisomer, tautomer, or isotopic variant thereof, and in particular aspects the compound is GAL475 (1-(2,6-Bis-methylamino-8-propylamino-pyrimido[5,4-d]pyrimidin-4-yl amino)-2-methyl-propan-2-ol), and a pharmaceutically acceptable salt, solvate, hydrate, enantiomer, stereoisomer, tautomer, or isotopic variant thereof, in the manufacture of a medicament for treating hyperphagia in a subject in need thereof.

In one aspect, the present disclosure includes use of a compound of formula (I) or a pharmaceutically acceptable salt, solvate, hydrate, enantiomer, stereoisomer, tautomer, or isotopic variant thereof, and in particular aspects the compound is GAL475 (1-(2,6-Bis-methylamino-8-propylamino-pyrimido[5,4-d]pyrimidin-4-yl amino)-2-methyl-propan-2-ol), and a pharmaceutically acceptable salt, solvate, hydrate, enantiomer, stereoisomer, tautomer, or isotopic variant thereof, in the manufacture of a medicament for treating hyperlipidemia in a subject in need thereof.

In one aspect, the present disclosure includes use of a compound of formula (I) or a pharmaceutically acceptable salt, solvate, hydrate, enantiomer, stereoisomer, tautomer, or isotopic variant thereof, and in particular aspects the compound is GAL475 (1-(2,6-Bis-methylamino-8-propylamino-pyrimido[5,4-d]pyrimidin-4-yl amino)-2-methyl-propan-2-ol), and a pharmaceutically acceptable salt, solvate, hydrate, enantiomer, stereoisomer, tautomer, or isotopic variant thereof, in the manufacture of a medicament for treating insulin resistance in a subject in need thereof.

Other features and characteristics of the subject matter of this disclosure, as well as the methods of operation, functions of related elements of structure and the combination of parts, and economies of manufacture, will become more apparent upon consideration of the following description, drawings, and the appended claims, all of which form a part of this specification.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1 shows a graph of body weight gain in rats after 28 days GAL475 administration compared to vehicle treated rats.

FIG. 2 shows food consumption in rats after GAL475 administration in % of consumption in the control group treated with vehicle.

FIG. 3 shows body weight gain in dogs after 28 days GAL475 administration at dosages of 30 mg/kg, 15 mg/kg, and 7.5 mg/kg as weight change percentage compared to vehicle.

FIG. 4 shows gastric content retention in rats after GAL475 administration compared to vehicle.

FIG. 5 shows individual change in body weight after 1 week in human subjects following a single oral dose of 450 mg GAL475 (left) versus placebo (right).

### DETAILED DESCRIPTION

While aspects of the subject matter of the present disclosure may be embodied in a variety of forms, the following description is merely intended to disclose some of these forms as specific examples of the subject matter encompassed by the present disclosure. Accordingly, the subject matter of this disclosure is not intended to be limited to the forms or embodiments so described.

GAL-475 (1-(2,6-Bis-methylamino-8-propylamino-pyrimido[5,4-d]pyrimidin-4-yl amino)-2-methyl-propan-2-ol) is an investigational drug that was initially developed as a breathing control modulator for the treatment of central and obstructive sleep apnea, the two major phenotypes of sleep disordered breathing. A description of the molecule and related compounds is found in patent #U.S. Ser. No. 10/294,228B2, which is incorporated herein by reference.

This breathing control modulation activity is likely mediated via the carotid body, a peripheral polymodal chemosensory organ, located at the carotid bifurcation, responsible for sensing changes in partial pressures of O₂, CO₂ or pH and activating the brainstem respiratory center to produce hyperventilation. As shown in the examples below, the present disclosure provides the surprising and unexpected use of GAL475 compounds for uses in treating metabolic syndromes and related disorders and conditions. The present disclosure includes disclosures and examples demonstrating that GAL475 administration provides slower gastric emptying and a consequent decrease in appetite. For example, as shown in Example 2, rats treated with GAL475 (40 mg/Kg body weight per day) for 28 days experienced a meaningful weight loss compared to vehicle in a dose dependent manner. As a further example, as shown in Example 3, dogs treated with GAL475 for 28 days experienced a 25% decrease in body weight and a dramatic decrease in food consumption compared to vehicle in a dose dependent manner. And as shown in Example 5, administration of GAL475 to human subjects indicated weight loss in humans as well. GAL475 can effectively lead to weight loss and to a decrease in appetite in humans as well as animals. The present disclosure includes using GAL475 for the treatment of metabolic syndromes, food intake disorders, hyperphagia, hyperlipidemia, insulin resistance, obesity, diabetes, and for effecting weight loss in humans.

### Definitions

Unless otherwise defined, all technical and scientific terms used herein have the same meaning as commonly understood by one of ordinary skill in the art to which this invention belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety.

The terms “a” and “an” refer to one or more (i.e., at least one) of the grammatical object of the article. By way of example, “a cell” encompasses one or more cells.

The term “treating”, or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilizing (i.e., not worsening) the state of disease, delaying or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. In addition to being useful as methods of treatment, the methods described herein may be useful for the prevention or prophylaxis of disease.

The phrases “co-administering” or “administering in combination” as used herein mean that two (or more) agents are administered in temporal juxtaposition. The co-administration or combination may including mixing the two agents into a single formulation, or by administering the two agents separately but simultaneously, or separately and within a short time of each other. For example, in general the two agents are co-administered within the time range of 0.5-168 hours.

Concentrations, amounts, times, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.01 to 2.0” should be interpreted to include not only the explicitly recited values of about 0.01 to about 2.0, but also include individual values and sub-ranges within the indicated range. Thus, included in this numerical range are individual values such as 0.5, 0.7, and 1.5, and sub-ranges such as from 0.5 to 1.7, 0.7 to 1.5, and from 1.0 to 1.5, etc. Furthermore, such an interpretation should apply regardless of the breadth of the range or the characteristics being described. Additionally, it is noted that all percentages are in weight, unless specified otherwise.

In understanding the scope of the present disclosure, the terms “including” or “comprising” and their derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps. It is understood that reference to any one of these transition terms (i.e., “comprising,” “consisting,” or “consisting essentially”) provides direct support for replacement to any of the other transition term not specifically used. For example, amending a term from “comprising” to “consisting essentially of” would find direct support due to this definition. Based on this definition, any element disclosed herein or incorporated by reference may be included in or excluded from the claimed invention.

As used herein, the terms “substantially” and “substantial” refer to a considerable degree or extent. When used in conjunction with, for example, an event, circumstance, characteristic, or property, the terms can refer to instances in which the event, circumstance, characteristic, or property occurs precisely as well as instances in which the event, circumstance, characteristic, or property occurs to a close approximation, such as accounting for typical tolerance levels or variability of the examples described herein.

As used herein, the term “about” is used to provide flexibility to a numerical range endpoint by providing that a given value may be “a little above” or “a little below” the endpoint. The degree of flexibility of this term can be dictated by the particular variable and would be within the knowledge of those skilled in the art to determine based on experience and the associated description herein. For example, in one aspect, the degree of flexibility can be within about ±10% of the numerical value. In another aspect, the degree of flexibility can be within about ±5% of the numerical value. In a further aspect, the degree of flexibility can be within about ±2%, ±1%, or ±0.05%, of the numerical value.

Generally herein, the term “or” includes “and/or.”

As used herein, a plurality of compounds, elements, or steps may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

The term “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a more concrete fashion.

As used herein, “modified-release” may include extended-release (XR), long-acting (LA), sustained release (SR), controlled-release (CR), delayed release (DR), enterically coated, repeat action, pulsatile release, or a combination thereof.

In certain embodiments, the composition includes an enteric coating. In other embodiments, the compound is contained in a pharmaceutically suitable capsule. In other embodiments, the capsule contains granules or powder of the compound, or an admixture of the compound with the carrier or excipient. In yet other embodiments, the excipient comprises a binder, disintegrant, diluent, buffer, lubricant, glidant, antioxidant, antimicrobial preservative, colorant, or flavorant. In yet other embodiments, the capsule is enterically coated but the granules or powders of the compound are not enterically coated. In yet other embodiments, the granules or powders of the compound are coated with an enteric coating before being placed into the capsule. In yet other embodiments, the granules or powders of the compound are coated with a plurality of enteric coatings, as to provide delivery of drug to different regions of the intestine of the subject. In yet other embodiments, at least a portion of the granules or powders of the compound are enterically coated. In yet other embodiments, the capsule is coated with an enteric coating that is different from the enteric coating that coats the granules or powders of the compound.

In certain embodiments, the compound is coated onto a base particles so as to form a core. In other embodiments the base particle is not enterically coated and the composition is contained in a pharmaceutically acceptable capsule that is enterically coated. In other embodiments, the core is coated with an enteric coating, thereby forming an enterically coated bead. In yet other embodiments, the enterically coated bead is contained in a pharmaceutically acceptable capsule. In yet other embodiments, the capsule contains beads coated with a plurality of enteric coatings, so that the capsule provides delivery of the compound to different regions of the intestine of the subject. In yet other embodiments, the contents of the capsule are dissolved or suspended in a pharmaceutically acceptable liquid as to provide a liquid-filled capsule. In yet other embodiments, the capsule is enterically coated but the liquid formulate on contained within does not comprise an enteric coating.

Furthermore, certain compositions, elements, excipients, ingredients, disorders, conditions, properties, steps, or the like may be discussed in the context of one specific embodiment or aspect or in a separate paragraph or section of this disclosure. It is understood that this is merely for convenience and brevity, and any such disclosure is equally applicable to and intended to be combined with any other embodiments or aspects found anywhere in the present disclosure and claims, which all form the application and claimed invention at the filing date. For example, a list of method steps, active agents, kits, or compositions described with respect to a compound of formula (I) or a pharmaceutically acceptable salt, solvate, hydrate, enantiomer, stereoisomer, tautomer, or isotopic variant thereof, and in particular aspects GAL475 (and related compounds described herein) or method of treating a certain subject is intended to and does find direct support for embodiments related to compositions, formulations of a compound of formula (I) or a pharmaceutically acceptable salt, solvate, hydrate, enantiomer, stereoisomer, tautomer, or isotopic variant thereof, and in particular aspects GAL475 (and related compounds described herein) or methods of treating a certain subject described in any other part of this disclosure, even if those method steps, active agents, kits, or compositions are not re-listed in the context or section of that embodiment or aspect.

Also as used herein, reference to “a” compound, salt or stereoisomer of GAL475 is intended to encompass “one or more” such compound including salts, solvates, hydrates, enantiomers, stereoisomers, tautomers, or isotopic variant thereof. Furthermore, reference to a “compound”, as in the discussion below of pharmaceutical formulations, is also intended to include salts, solvates, hydrates, enantiomers, stereoisomers, tautomers, or isotopic variant of the compound.

The compounds of the invention may possess one or more stereocenters, and each stereocenter may exist independently in either the (R) or (S) configuration. In certain embodiments, compounds described herein are present in optically active or racemic forms. The compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound of the invention, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compounds of the invention exist as tautomers. All tautomers are included within the scope of the compounds recited herein.

In certain embodiments, compounds described herein are prepared as prodrugs. A “prodrug” is an agent converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compounds of the invention are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I ¹³N, ¹⁵N, ¹⁵O, ¹⁷O, ¹⁸O, ³²P, and ³⁵S. In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵0 and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

Methods of synthesis of the molecules of the present disclosure are disclosed, for example, in U.S. Pat. No. 10,294,228, which is incorporated herein by reference in its entirety for all purposes.

### Pharmaceutical Compositions and Formulations

The invention also encompasses the use of pharmaceutical compositions of at least one compound of the invention or a salt, solvate, enantiomer, diastereoisomer or tautomer thereof to practice the methods of the invention. Such a pharmaceutical composition may consist of at least one compound of the invention or a salt, solvate, enantiomer, diastereoisomer or tautomer thereof, in a form suitable for administration to a subject, or the pharmaceutical composition may comprise at least one compound of the invention or a salt, solvate, enantiomer, diastereoisomer or tautomer thereof, and one or more pharmaceutically acceptable carriers, one or more additional ingredients, or some combination of these. The at least one compound of the invention may be present in the pharmaceutical composition in the form of a physiologically acceptable salt, such as in combination with a physiologically acceptable cation or anion, as is well known in the art.

In an embodiment, the pharmaceutical compositions useful for practicing the method of the invention may be administered to deliver a dose of between 1 ng/kg/day and 5000 mg/kg/day. In other embodiments, the pharmaceutical compositions useful for practicing the invention may be administered to deliver a dose of between 1 ng/kg/day and 1,000 mg/kg/day.

Effective dosage amounts provided in a composition may include dosage units containing about 0.01-500 mg/kg, about 1-100 mg/kg per day, or about 5-50 mg/kg per day of the one or more compounds of the present disclosure. In some aspects, dosage units are administered every other day, biweekly, or weekly.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the invention may be suitably developed for nasal, inhalational, oral, rectal, vaginal, pleural, peritoneal, parenteral, topical, transdermal, pulmonary, intranasal, buccal, ophthalmic, epidural, intrathecal, intravenous or another route of administration. A composition useful within the methods of the invention may be directly administered to the brain, the brainstem, or any other part of the central nervous system of a mammal or bird. Other contemplated formulations include projected nanoparticles, microspheres, liposomal preparations, coated particles, polymer conjugates, resealed erythrocytes containing the active ingredient, and immunologically-based formulations.

In certain embodiments, the compositions of the invention are part of a pharmaceutical matrix, which allows for manipulation of insoluble materials and improvement of the bioavailability thereof, development of immediate, controlled or sustained release products, and generation of homogeneous compositions. By way of example, a pharmaceutical matrix may be prepared using hot melt extrusion, solid solutions, solid dispersions, size reduction technologies, molecular complexes (e.g. cyclodextrins, and others), microparticulate, and particle and formulation coating processes. Amorphous or crystalline phases may be used in such processes.

The route(s) of administration will be readily apparent to the skilled artisan and will depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology and pharmaceutics. In general, such preparatory methods include the step of bringing the active ingredient into association with a carrier or one or more other accessory ingredients, and then, if necessary or desirable, shaping or packaging the product into a desired single-dose or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

In certain embodiments, the compositions of the invention are formulated using one or more pharmaceutically acceptable excipients or carriers. In certain embodiments, the pharmaceutical compositions of the invention comprise a therapeutically effective amount of at least one compound of the invention and a pharmaceutically acceptable carrier. Pharmaceutically acceptable carriers, which are useful, include, but are not limited to, glycerol, water, saline, ethanol, recombinant human albumin (e.g. Recombumin®), solubilized gelatins (e.g. Gelofusine®), and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

According to the principles of the present invention, the compound of the present disclosure may be present in the composition in the form of free base or in the form of its salts or mixtures thereof. Salts according to the principles of the present invention may be prepared by, for example, reacting the free base forms with one or more equivalents of the appropriate base or acid, respectively, in a solvent or medium in which the salt is insoluble, or in a solvent such as water which is then removed in vacuo, by freeze-drying, or by exchanging the ions of an existing salt for another ion on a suitable ion exchange resin. Each possibility represents a separate embodiment of the invention.

The carrier may be a solvent or dispersion medium containing, for example, water, ethanol, polyol (for example, glycerol, propylene glycol, and liquid polyethylene glycol, and the like), recombinant human albumin, solubilized gelatins, suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions may be brought about by including in the composition an agent that delays absorption, for example, aluminum monostearate or gelatin.

The compositions of the present invention may further comprise one or more pharmaceutically acceptable excipient(s) selected from, but not limited to, co-surfactants/solubilizers, solvents/co-solvents, water immiscible solvents, water, water miscible solvents, oily components, hydrophilic solvents, emulsifiers, preservatives, antioxidants, anti-foaming agents, stabilizers, buffering or pH adjusting agents, osmotic agents, pore forming agents, osmotic adjustment agents, or any other excipient known in the art. Each possibility represents a separate embodiment. Suitable co-surfactants or solubilizers include, but are not limited to, polyethylene glycols, polyoxyethylene-polyoxypropylene block copolymers known as “poloxamer”, polyglycerin fatty acid esters such as decaglyceryl monolaurate and decaglyceryl monomyristate, sorbitan fatty acid ester such as sorbitan monostearate, polyoxyethylene sorbitan fatty acid ester such as polyoxyethylene sorbitan monooleate (Tween), polyethylene glycol fatty acid ester such as polyoxyethylene monostearate, polyoxyethylene alkyl ether such as polyoxyethylene lauryl ether, polyoxyethylene castor oil and hardened castor oil such as polyoxyethylene hardened castor oil, and the like or mixtures thereof. Each possibility represents a separate embodiment of the invention. Suitable solvents/co-solvents include, but not limited to, alcohols, triacetin, dimethyl isosorbide, glycofurol, propylene carbonate, water, dimethyl acetamide, and the like or mixtures thereof. Each possibility represents a separate embodiment of the invention. Suitable anti-foaming agents include, but are not limited to, silicon emulsions or sorbitan sesquioleate. Each possibility represents a separate embodiment of the invention. Suitable stabilizers to prevent or reduce the deterioration of the components in the compositions of the present invention include, but are not limited to, antioxidants such as glycine, α-tocopherol or ascorbate, BHA, BHT, and the like or mixtures thereof. Each possibility represents a separate embodiment of the invention. Suitable tonicity modifiers include, but are not limited to, mannitol, sodium chloride, and glucose. Each possibility represents a separate embodiment of the invention. Suitable buffering agents include, but are not limited to, acetates, phosphates, and citrates with suitable cations. Each possibility represents a separate embodiment of the invention.

Formulations may be employed in admixtures with conventional excipients, i.e., pharmaceutically acceptable organic or inorganic carrier substances suitable for oral, parenteral, nasal, inhalational, intravenous, subcutaneous, transdermal enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or fragrance-conferring substances and the like. They may also be combined where desired with other active agents, e.g., other analgesic, anxiolytics or hypnotic agents. As used herein, “additional ingredients” include, but are not limited to, one or more ingredients that may be used as a pharmaceutical carrier.

The composition of the invention may comprise a preservative from about 0.005% to 2.0% by total weight of the composition. The preservative is used to prevent spoilage in the case of exposure to contaminants in the environment. Examples of preservatives useful in accordance with the invention include but are not limited to those selected from the group consisting of benzyl alcohol, sorbic acid, parabens, imidurea (imidazolidinyl urea) and combinations thereof. A particularly preferred preservative is a combination of about 0.5% to 2.0% benzyl alcohol and 0.05% to 0.5% sorbic acid.

The composition preferably includes an antioxidant and a chelating agent which inhibit the degradation of the compound. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition which may be detrimental to the shelf life of the formulation. While BHT and disodium edetate are the particularly preferred antioxidant and chelating agent, respectively, for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, acacia, and ionic or non-ionic surfactants. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, ionic and non-ionic surfactants, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying. Methods for mixing components include physical milling, the use of pellets in solid and suspension formulations and mixing in a transdermal patch, as known to those skilled in the art.

### Administration/Dosing

The regimen of administration may affect what constitutes an effective amount. The therapeutic formulations may be administered to the patient either prior to or after the onset of a breathing disorder event. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat a breathing control disorder in the patient. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 0.01 mg/kg to 100 mg/kg of body weight/per day. One of ordinary skill in the art would be able to study the relevant factors and make the determination regarding the effective amount of the therapeutic compound without undue experimentation.

The compound may be administered to an animal as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose will be readily apparent to the skilled artisan and will depend upon any number of factors, such as, but not limited to, the type and severity of the disease being treated, the type and age of the animal, etc.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of breathing disorders in a patient.

In certain embodiments, the compositions of the invention are administered to the patient in dosages that range from one to five times per day or more. In other embodiments, the compositions of the invention are administered to the patient in range of dosages that include, but are not limited to, once every day, every two days, every three days to once a week, and once every two weeks. It will be readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any patient will be determined by the attending physician taking all other factors about the patient into account.

Compounds of the invention for administration may be in the range of from about 1 μg to about 7,500 mg, about 20 μg to about 7,000 mg, about 40 μg to about 6,500 mg, about 80 μg to about 6,000 mg, about 100 μg to about 5,500 mg, about 200 μg to about 5,000 mg, about 400 μg to about 4,000 mg, about 800 μg to about 3,000 mg, about 1 mg to about 5,500 mg, about 2 mg to about 5,000 mg, about 10 mg to about 4,000 mg, about 15 mg to about 3,750 mg, about 20 mg to about 3000 mg, about 30 mg to about 2500 mg, about 40 mg to about 2000 mg, about 50 mg to about 1500 mg, about 60 mg to about 1000 mg, about 70 mg to about 900 mg, about 80 mg to about 800 mg, and any and all whole or partial increments there-in-between.

In some embodiments, the dose of a compound of the invention is from about 0.5 μg and about 5,000 mg. In some embodiments, a dose of a compound of the invention used in compositions described herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of breathing disorder in a patient.

The term “container” includes any receptacle for holding the pharmaceutical composition or for managing stability or water uptake. For example, in certain embodiments, the container is the packaging that contains the pharmaceutical composition, such as liquid (solution and suspension), semisolid, lyophilized solid, solution and powder or lyophilized formulation present in dual chambers. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing a breathing disorder in a patient.

### Administration

Routes of administration of any of the compositions of the invention include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, epidural, intrapleural, intraperitoneal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, emulsions, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

The present invention provides a method for treating or delaying the progression or onset of diabetes, especially type-2 diabetes, including complications of diabetes, such as retinopathy, neuropathy, nephropathy and delayed wound healing, and related diseases such as insulin resistance (impaired glucose homeostasis), hyperglycemia, hyperinsulinemia, elevated blood levels of fatty acids or glycerol, overweight, obesity, hyperlipidemia including hypertriglyceridemia, Syndrome X, atherosclerosis, hypertension, and cardiovascular diseases and events such as coronary heart disease, cerebrovascular disease, peripheral arterial disease, rheumatic heart disease, congenital heart disease, deep vein thrombosis and pulmonary embolism, non-fatal myocardial infarction, or non-fatal stroke, and for increasing high density lipoprotein levels.

Obesity involves excess body fat accumulation and people are generally defined to be obese when their body mass index (BMI; a value of body mass (kg) over body height squared (m)) is 30 or higher. Obesity is most commonly caused by energy imbalance due to excessive food intake compared to energy consumption over a long period of time (“positive energy balance”). Obesity, being a metabolic disease that affects the entire body, increases the possibility of developing of diabetes and hyperlipidemia, increases the risk of the incidence of sexual dysfunction, arthritis, and cardiovascular disease, and is associated with cancer development in some cases.

An overweight condition involves moderate body fat accumulation and people are generally defined to be overweight when their body mass index (BMI; a value of body mass (kg) over body height squared (m)) is 25 to 29.5. Overweightness is most commonly caused by energy imbalance due to excessive food intake compared to energy consumption over a long period of time. Overweightness increases the mortality and overtakes smoking as a primary cause of cancer in developing countries. Overweightness also increases the risk of oligospermia and azoospermia and physiological well-being.

The term “treating” as used herein with reference to obesity and an overweight condition refers to preventing weight gain, promoting weight loss, reducing excess body weight or treating obesity/overweight (e.g. by control of appetite, feeding, food intake, calorie intake, and/or energy expenditure), including morbid obesity, as well as diseases and health conditions associated with excessive weight gain including, but not limited to, inflammation, gallbladder disease and sleep apnea.

### Oral Administration

In one embodiment, compounds of the invention may be formulated to prepare a pharmaceutical composition for oral administration. In further embodiments, the composition for oral administration may be designed to promote a modified release of the drug, such that the location, extent and rate of exposure of the compound when ingested are modulated. Factors that affect the target zone for exposure of a drug may be the drug's pH or enzymatic stability, reactivity with other drugs (e.g., certain antibiotics), solubility as a salt or free base, ionization behavior, and pharmacodynamic and pharmacokinetic behaviors in specific environments. Some drugs are better absorbed in the duodenum or other intestinal locations.

Delayed release is a particularly useful mode of modified release that delivers drug in its most concentrated form to the duodenum or other intestinal location. In a preferred embodiment, compounds of the present invention are formulated to promote delivery to the duodenum and, optionally, other intestinal locations. Delayed release may be achieved using compositions that include enteric coatings. Enteric coatings are insoluble in highly acidic environments, with the polyacidic coating remaining non-ionized and intact at gastric pH. However, under mildly acidic (>pH 5.5), neutral or mildly alkaline conditions (pH 6.5-7.6) of the duodenum or other intestinal regions, the coating ionizes, swells and breaks down, exposing the coated entity to the environment. Coating options exist to allow ionization at or near a specific pH (e.g. Eudragit L-110, ionization threshold pH 6.0; Eudragit S-100, ionization threshold pH 7.0).

In a further embodiment, compounds of the present invention may be formulated with an enteric coating which has been modified by adding plasticizers to the polymer before coating. The plasticizers may be added to adjust resistance to chipping or cracking of the coating, while also lowering the glass transition temperature of the coating to enable smooth and even spreadability of the coating during its application. Suitable plasticizers include polyethylene glycol 8000 (PEG 8000), triethyl citrate (TEC), and triacetin, which may be incorporated into the polymeric enteric coating agent.

Compounds of the present invention may be enterically formulated under a variety of dosage forms, including (but not limited to) capsules, granules of the active drug itself, beads, and tablets. In one embodiment, the composition may comprise a drug encapsulated in a capsule that is enterically coated to release the drug in the duodenum or other intestinal environment. In one aspect of the invention, pharmaceutically acceptable capsules include hard capsules, which may be composed of plant derived polysaccharides, starches, and cellulose, or gelatin. In another embodiment, pharmaceutically acceptable capsules include soft gelatin capsules. The gelatin capsule may be composed of animal-derived collagen or from a Hypromellose (hydroxypropyl methylcellulose), a modified form of cellulose, and manufactured using optional mixtures of gelatin, water and plasticizers such as sorbitol or glycerol

In one embodiment, molecules of the invention may be encapsulated in pure granular or powdered form, with no carriers, excipients or other pharmaceutically acceptable additives. In other embodiments, molecules of the invention may be encapsulated together with one or more pharmaceutically acceptable carriers, excipients, antioxidants (e.g., sodium metabisulfite, butylated hydroxy toluene [BHT]), antifungals, (e.g., benzoic and ascorbic acids and their salts, and phenolic compounds such as methyl, ethyl, propyl and butyl p-hydroxybenzoate (parabens)), antimicrobial preservatives (e.g., sodium benzoate, sorbic acid), colorants, and flavorants. The excipients may aid in capsule-filling behavior, stability, and in the distribution of the drug when the capsule disintegrates in the body. In another embodiment, granules and/or powders of compounds of the present invention may be enterically coated before being placed in a capsule. The enterically coated granules and/or powders placed in the capsule may feature one or several types of enteric coating to enable delivery of the drug to different regions of the intestine. The capsule may lack enteric coating or may be coated with an enteric coating matching or differing entirely from the coating applied to any of the enterically coated material inside the capsule.

In a further embodiment, molecules of the invention may be encapsulated in a liquid in the form of a solution or suspension in water or various pharmaceutically acceptable oils or other dispersion medium (e.g., mineral oil, sesame oil, safflower oil, coconut oils), optionally with such excipients as co-solvents (e.g., propylene glycol, glycerol), solubility enhancers (e.g., sorbitol, dextrose), wetting agents (e.g.; polysorbates [Tweens], sorbitan esters [Span], hydrophobic colloids [cellulose derivatives], thickening agents (e.g., methylcellulose, microcrystalline cellulose), buffers (e.g., disodium hydrogen phosphate), antioxidants (e.g., butylated hydroxy toluene [BHT], citric acid, potassium sorbate), antifungals (e.g., benzoic and ascorbic acids and their salts, and phenolic compounds such as methyl, ethyl, propyl and butyl p-hydroxybenzoate (parabens)) antimicrobial preservatives (e.g., sodium benzoate, sorbic acid), colorants and flavorants. In some embodiments, compounds of the present invention may be formulated for liquid filled capsules in the form of the pure drug as granules and/or powders in the liquid. In a related embodiment, the capsule contained the drug in liquid may be enterically coated. In another embodiment, granules and/or powders of compounds of the invention may be enterically coated before being placed in a liquid and the combination placed in a capsule. The enterically coated granules and/or powder may feature one or several types of enteric coating to enable deliver of the drug to different regions of the intestine. The capsule may lack enteric coating or may be coated with an enteric coating matching or differing entirely from the coating applied to any of the enterically coated material inside the capsule.

In another embodiment, molecules of the present invention may be encapsulated in a capsule comprised of material which affords post-gastric drug delivery without the need for the separate application of an enteric coating (e.g., Entericare enteric softgels). The molecules may be encapsulated in such capsules as granules or powders with or without excipients, and as solutions or suspensions as described above.

In some embodiments, the solid particles of the compounds of the present invention, as a variety of particle sizes and particle size distributions, may be admixed with excipients such as microcrystalline cellulose or lactose and formed as a bead which comprise the drug-containing core onto which the enteric coating is applied. In some embodiments, molecules of the current invention may be formed as a suspension or solution including, optionally, buffers (e.g., aq. 1 N HCl with tris-hydroxymethyl-aminomethane [TRIS]), and binders (e.g., Opadry Clear Coat Powder) and coated onto a base particle, for example sugar beads (e.g., Sugar Spheres, NF particles) to form a bead. In another embodiment, the beads may be enterically coated. In yet another embodiment, molecules of the invention may be formulated as enterically coated beads, as described above, and the beads further formulated by encapsulation. In a further embodiment, a combination of beads with different types of enteric coating may be encapsulated, such that once released from the capsule, compounds of the invention are made available in a controlled manner at different regions ranging from the duodenum to other parts of the intestine. The capsule may lack enteric coating or may be coated with an enteric coating matching or differing entirely from the coating applied to any of the enterically coated material inside the capsule.

In a further embodiment, compounds of the present invention may be formulated as tablets or caplets which alone or in combination with other formulation components deliver drug to the duodenum or other intestinal region. In one embodiment, compounds of the invention are formulated as tablets or caplets which are enterically coated and which constitute the dosage form administered. In another embodiment, tablets or caplets of suitable size and shape may be placed inside a capsule. In one such embodiment, the capsule may be enterically coated and contain non-enterically coated tablets or caplets which are released from the capsule in the duodenum or other intestinal region. In yet another such embodiment, the capsule may be designed to disintegrate in the stomach and release enterically coated tablets or caplets for subsequent delivery to duodenum or other intestinal regions. In yet another such embodiment, the capsule and tablets or caplets contained within may both be enterically coated to provide further control over the release of the tablets or caplets from the capsule, and the subsequent release of the drug from the tablet or caplet. In a further related embodiment, tablets or caplets featuring a variety of enteric coating may combined and place in a capsule which itself may optionally be enterically coated as well. Materials which are useful for enteric coatings for tablets and caplets include but are not limited to those described above for application to capsules.

Enteric coatings may permit premature drug release in acidic media. In a still further embodiment, compounds of the present invention may be formulated such that a subcoating is applied before the enteric coating is applied. The subcoating may comprise application to the enteric substrate of a soluble subcoating agent, examples of which are hydroxypropylmethylcellulose, povidone, hydroxypropyl cellulose, polyethylene glycol 3350, 4500, 8000, methyl cellulose, pseudo ethylcellulose and amylopectin. A thin subcoating layer on the enteric substrate impedes water penetration through the enteric coating on the capsule shell or into the core where the active ingredient is located, preventing premature drug release. The subcoating may also promote the release of the drug in a basic environment by moderating the acidic microenvironment at the interface between the core and the enteric coating. In some embodiments, compounds of the present invention are formulated with a subcoating containing organic acids intended to promote more rapid polymer dissolution of a capsule as the coating degrades in environments with pH 5-6, promoting a rapid release of the drug in basic media.

For oral application, particularly suitable are tablets, dragees, liquids, drops, capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The compositions intended for oral use may be prepared according to any method known in the art and such compositions may contain one or more agents selected from the group consisting of inert, non-toxic, generally recognized as safe (GRAS) pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically controlled release tablets. Tablets may further comprise a sweetening agent, a flavoring agent, a coloring agent, a preservative, or some combination of these in order to provide for pharmaceutically elegant and palatable preparation. Hard capsules comprising the active ingredient may be made using a physiologically degradable composition, such as gelatin. The capsules comprise the active ingredient, and may further comprise additional ingredients including, for example, an inert solid diluent such as calcium carbonate, calcium phosphate, or kaolin.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin from animal-derived collagen or from a hypromellose, a modified form of cellulose, and manufactured using optional mixtures of gelatin, water and plasticizers such as sorbitol or glycerol. Such soft capsules comprise the active ingredient, which may be mixed with water or an oil medium such as peanut oil, liquid paraffin, or olive oil.

For oral administration, the compounds of the invention may be in the form of tablets or capsules prepared by conventional means with pharmaceutically acceptable excipients such as binding agents; fillers; lubricants; disintegrates; or wetting agents. If desired, the tablets may be coated using suitable methods and coating materials such as OPADRY™ film coating systems available from Colorcon, West Point, Pa. (e.g., OPADRY™ OY Type, OYC Type, Organic Enteric OY—P Type, Aqueous Enteric OY-A Type, OY-PM Type and OPADRY™ White, 32K18400). It is understood that similar type of film coating or polymeric products from other companies may be used.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycolate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e., having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e., drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

The present invention also includes a multi-layer tablet comprising a layer providing for the delayed release of one or more compounds useful within the methods of the invention, and a further layer providing for the immediate release of one or more compounds useful within the methods of the invention. Using a wax/pH-sensitive polymer mix, a gastric insoluble composition may be obtained in which the active ingredient is entrapped, ensuring its delayed release.

Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl para-hydroxy benzoates or sorbic acid). Liquid formulations of a pharmaceutical composition of the invention which are suitable for oral administration may be prepared, packaged, and sold either in liquid form or in the form of a dry product intended for reconstitution with water or another suitable vehicle prior to use.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Injectable formulations may also be prepared, packaged, or sold in devices such as patient-controlled analgesia (PCA) devices. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3-butanediol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form in a recombinant human albumin, a fluidized gelatin, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Topical Administration

An obstacle for topical administration of pharmaceuticals is the stratum corneum layer of the epidermis. The stratum corneum is a highly resistant layer comprised of protein, cholesterol, sphingolipids, free fatty acids and various other lipids, and includes cornified and living cells. One of the factors that limit the penetration rate (flux) of a compound through the stratum corneum is the amount of the active substance that can be loaded or applied onto the skin surface. The greater the amount of active substance which is applied per unit of area of the skin, the greater the concentration gradient between the skin surface and the lower layers of the skin, and in turn the greater the diffusion force of the active substance through the skin. Therefore, a formulation containing a greater concentration of the active substance is more likely to result in penetration of the active substance through the skin, and more of it, and at a more consistent rate, than a formulation having a lesser concentration, all other things being equal.

Formulations suitable for topical administration include, but are not limited to, liquid or semi-liquid preparations such as liniments, lotions, oil-in-water or water-in-oil emulsions such as creams, ointments or pastes, and solutions or suspensions. Topically administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

Enhancers of permeation may be used. These materials increase the rate of penetration of drugs across the skin. Typical enhancers in the art include ethanol, glycerol monolaurate, PGML (polyethylene glycol monolaurate), dimethylsulfoxide, and the like. Other enhancers include oleic acid, oleyl alcohol, ethoxydiglycol, laurocapram, alkanecarboxylic acids, dimethylsulfoxide, polar lipids, or N-methyl-2-pyrrolidone.

One acceptable vehicle for topical delivery of some of the compositions of the invention may contain liposomes.

In alternative embodiments, the topically active pharmaceutical composition may be optionally combined with other ingredients such as adjuvants, anti-oxidants, chelating agents, surfactants, foaming agents, wetting agents, emulsifying agents, viscosifiers, buffering agents, preservatives, and the like. In other embodiments, a permeation or penetration enhancer is included in the composition and is effective in improving the percutaneous penetration of the active ingredient into and through the stratum corneum with respect to a composition lacking the permeation enhancer. Various permeation enhancers, including oleic acid, oleyl alcohol, ethoxydiglycol, laurocapram, alkanecarboxylic acids, dimethylsulfoxide, polar lipids, or N-methyl-2-pyrrolidone, are known to those of skill in the art. In another aspect, the composition may further comprise a hydrotropic agent, which functions to increase disorder in the structure of the stratum corneum, and thus allows increased transport across the stratum corneum. Various hydrotropic agents such as isopropyl alcohol, propylene glycol, or sodium xylene sulfonate, are known to those of skill in the art.

The topically active pharmaceutical composition should be applied in an amount effective to affect desired changes. As used herein “amount effective” shall mean an amount sufficient to cover the region of skin surface where a change is desired. An active compound should be present in the amount of from about 0.0001% to about 15% by weight volume of the composition. More preferable, it should be present in an amount from about 0.0005% to about 5% of the composition; most preferably, it should be present in an amount of from about 0.001% to about 1% of the composition. Such compounds may be synthetically—or naturally derived.

### Buccal Administration

A pharmaceutical composition of the invention may be prepared, packaged, or sold in a formulation suitable for buccal administration. Such formulations may, for example, be in the form of tablets or lozenges made using conventional methods, and may contain, for example, 0.1 to 20% (w/w) of the active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein. The examples of formulations described herein are not exhaustive and it is understood that the invention includes additional modifications of these and other formulations not described herein, but which are known to those of skill in the art.

### Rectal Administration

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants, and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants, and preservatives.

### Additional Administration Forms

Additional dosage forms of this invention include dosage forms as described in U.S. Pat. Nos. 6,340,475, 6,488,962, 6,451,808, 5,972,389, 5,582,837, and 5,007,790, U.S. Patent Applications Nos. 20030147952, 20030104062, 20030104053, 20030044466, 20030039688, and 20020051820, PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757, the disclosures of which are incorporated herein by reference in their entireties.

### Controlled Release Formulations and Drug Delivery Systems

In certain embodiments, the composition is designed to promote controlled release of the drug, such that the location, extent and rate of exposure of the compound when administered are modulated. Factors that affect the target zone for exposure of an orally administered drug may be the drug's pH and enzymatic stability, reactivity with other drugs (e.g., certain antibiotics), solubility as a salt or free base, ionization behavior, and pharmacodynamic and pharmacokinetic behaviors in specific environments.

Controlled- or sustained-release formulations of a pharmaceutical composition of the invention may be made using conventional technology. In some cases, the dosage forms to be used can be provided as slow or controlled-release of one or more active ingredients therein using, for example, hydroxypropyl methylcellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions of the invention. Thus, single unit dosage forms suitable for oral administration, such as tablets, capsules, gelcaps, and caplets that are adapted for controlled-release are encompassed by the present invention.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include targeted delivery within the gastrointestinal tract upon oral administration, extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example water, pH, temperature, enzymes, bacteria, or other physiological conditions or compounds. The term “controlled-release component” in the context of the present invention is defined herein as a compound or compounds, including, but not limited to, polymers, polymer matrices, gels, permeable membranes, liposomes, or microspheres or a combination thereof that facilitates the controlled-release of the active ingredient.

In certain embodiments, the formulations of the present invention may be, but are not limited to, short-term, rapid-offset, immediate release, as well as controlled, for example, sustained release, delayed release and pulsatile release formulations. The active drug substance can also be coated on an implantable medical device to be eluted or be released using a remotely activated system.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release that is longer that the same amount of agent administered in bolus form.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation (drug embedded in polymeric matrices).

In a preferred embodiment of the invention, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that may, although not necessarily, includes a delay of from about 10 minutes up to about 24 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 24 hours, about 12 hours, about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 24 hours, about 12 hours, about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

A drug may be better absorbed in the duodenum or other intestinal locations. A particularly useful mode of controlled release is one which minimizes release of drug in the stomach, while delivering drug in its most concentrated form to the duodenum or other intestinal locations. In certain embodiments, the compounds of the present invention are formulated to promote delivery to the duodenum and, optionally, other intestinal locations. Controlled release that delivers drug to the duodenum or other intestinal regions may be achieved using compositions that include enteric coatings. Enteric coatings are insoluble in highly acidic environments, often comprising a polyacidic coating that remains non-ionized and intact at gastric pH. However, under mildly acidic (>pH 5.5) or neutral or mildly alkaline conditions (pH 6.5-7.6) of the duodenum or other intestinal regions, the coating ionizes, swells and breaks down, exposing the coated entity to the environment. Coating options exist to allow ionization at or near a specific pH (e.g. Eudragit L-110, ionization threshold pH 6.0; Eudragit S-100, ionization threshold pH 7.0). It is understood that similar type or grade of film coating or polymeric products from other companies may be used.

In certain embodiments, compounds of the present invention are formulated with an enteric coating, which has been modified by adding plasticizers to the polymer before coating. The plasticizers may be added to adjust resistance to chipping or cracking of the coating, while also lowering the glass transition temperature of the coating to enable smoothness and even spreadability of the coating during its application. Suitable plasticizers include polyethylene glycol 8000 (PEG 8000), triethyl citrate (TEC), and triacetin, which may be incorporated into the polymeric enteric coating agent.

Compounds of the present invention may be enterically formulated under a variety of dosage forms, including (but not limited to) capsules, granules of the active drug itself, beads, micro spheres, and tablets. In certain embodiments, the composition comprises a drug encapsulated in a capsule enterically coated to release the drug in the duodenum or other intestinal environment. In other embodiments, pharmaceutically acceptable capsules include hard capsules. In yet other embodiments, pharmaceutically acceptable capsules include soft gelatin capsules.

In certain embodiments, a compound of the invention is encapsulated in pure granular or powdered form, with no carriers, excipients or other pharmaceutically acceptable additives. In other embodiments, a compound of the invention is encapsulated together with one or more pharmaceutically acceptable carriers, excipients, antioxidants, antifungals, (e.g., benzoic and ascorbic acids and their salts, and phenolic compounds such as methyl, ethyl, propyl and butyl p-hydroxybenzoate (parabens)), antimicrobial preservatives, colorants, and flavorants. The excipients may aid in capsule-filling behaviour, stability, and in the distribution of the drug when the capsule disintegrates in the body. In other embodiments, granules and/or powders of a compound of the present invention are enterically coated before being placed in a capsule. The enterically coated granules and/or powders placed in the capsule may feature one or several types of enteric coating to enable delivery of the drug to different regions of the intestine. The capsule may lack enteric coating or may be coated with an enteric coating that is the same as or distinct from the coating applied to any of the enterically coated materials inside the capsule.

In certain embodiments, a compound of the invention is encapsulated in a liquid in the form of a solution or suspension in water or various pharmaceutically acceptable oils or other dispersion medium, optionally with such excipients as co-solvents (e.g., PEG 300, PEG 400, propylene glycol, glycerol, tween 80, ethanol), solubility enhancers (e.g., sorbitol, dextrose), wetting agents (e.g., thickening agents), buffers (e.g., disodium hydrogen phosphate), antioxidants, antifungals, preservatives, colorants and flavorants. In certain embodiments, a compound of the present invention is formulated for liquid filled capsules in the form of the pure drug as granules and/or powders in the liquid. In other embodiments, the capsule containing the compound in liquid is enterically coated. In yet other embodiments, granules and/or powders of a compound of the invention are enterically coated before being placed in a liquid and the combination placed in a capsule. The enterically coated granules and/or powder may feature one or several types of enteric coating to enable delivery of the drug to distinct regions of the intestine. The capsule may lack enteric coating or may be coated with an enteric coating that is the same as or distinct from the coating applied to any of the enterically coated materials inside the capsule.

In certain embodiments, a compound of the present invention is encapsulated in a capsule comprised of material that affords post-gastric drug delivery without the need for the separate application of an enteric coating (e.g., Entericare enteric softgels). The compound may be encapsulated in such capsules as granules or powders with or without excipients, and as solutions or suspensions as described above.

In certain embodiments, the solid particles of a compound of the present invention, as a variety of particle sizes and particle size distributions, are admixed with excipients such as microcrystalline cellulose or lactose and formed as a bead that comprises the drug-containing core onto which the enteric coating is applied. In other embodiments, a compound of the present invention is formed as a suspension or solution including, optionally, buffers (e.g., aq. 1 N HCl with tris(hydroxymethyl)aminomethane “TRIS”), and binders (e.g., Opadry Clear Coat Powder) and coated onto a base particle, for example sugar beads (e.g., Sugar Spheres, NF particles) to form a bead. In yet other embodiments, the beads are enterically coated. In yet other embodiments, a compound of the invention is formulated as enterically coated beads, as described above, and the beads further formulated by encapsulation. In yet other embodiments, a combination of beads with different types of enteric coating is encapsulated, such that once released from the capsule, the compound of the invention is made available in a controlled manner at different regions ranging from the duodenum to other parts of the intestine. The capsule may lack enteric coating or may be coated with an enteric coating that is the same as or distinct from the coating applied to any of the enterically coated materials inside the capsule.

In certain embodiments, a compound of the present invention is formulated as tablets or caplets which alone or in combination with other formulation components deliver drug to the duodenum or other intestinal region. In other embodiments, a compound of the invention is formulated as tablets or caplets that are enterically coated and that constitute the dosage form administered. In yet other embodiments, tablets or caplets of suitable size and shape are placed inside a capsule. In yet other embodiments, the capsule is enterically coated and contains non-enterically coated tablets or caplets, which are released from the capsule in the duodenum or other intestinal region. In yet other embodiments, the capsule is designed to disintegrate in the stomach and release enterically coated tablets or caplets for subsequent delivery to duodenum or other intestinal regions. In yet other embodiments, the capsule and tablets or caplets contained within are both enterically coated to provide further control over the release of the tablets or caplets from the capsule, and the subsequent release of the drug from the tablet or caplet. In yet other embodiments, tablets or caplets featuring a variety of enteric coating are combined and placed in a capsule which itself may optionally be enterically coated as well. Materials useful for enteric coatings for tablets and caplets include but are not limited to those described above for application to capsules.

Enteric coatings may permit premature drug release in acidic media. In certain embodiments, a compound of the present invention is formulated such that a subcoating is applied before the enteric coating is applied. The subcoating may comprise application to the enteric substrate of a soluble subcoating agent, examples of which are hydroxypropylmethylcellulose, povidone, hydroxypropyl cellulose, polyethylene glycol 3350, 4500, 8000, methyl cellulose, pseudo ethylcellulose and amylopectin. It is understood that similar type of synthetic and semisynthetic polymeric products from other companies may be used. A thin subcoating layer on the enteric substrate impedes water penetration through the enteric coating on the capsule shell or into the core where the active ingredient is located, preventing premature drug release. The subcoating may also promote the release of the drug in a basic environment by moderating the acidic microenvironment at the interface between the core and the enteric coating. In certain embodiments, a compound of the present invention is formulated with a subcoating containing organic acids intended to promote more rapid polymer dissolution of a capsule as the coating degrades in environments with pH 5-6, promoting a rapid release of the drug in basic media.

The present invention also includes within its scope the preparation of compositions containing a compound of formula (I) or a pharmaceutically acceptable salt, solvate, hydrate, enantiomer, stereoisomer, tautomer, or isotopic variant thereof, and in particular aspects GAL475 wherein the compositions are useful as medicaments. The pharmaceutical compositions contain as an active substance a therapeutically effective amount of the compound of formula (I), including GAL475, as well as salts, solvates, hydrates, enantiomers, stereoisomers, tautomers, or isotopic variant thereof, in association with one or more pharmaceutically acceptable diluents, preservatives, solubilizers, emulsifiers, adjuvant, excipients and carriers conventionally used in pharmaceutical and veterinary formulations. The present pharmaceutical formulations can be adapted for administration to humans and/or animals.

A pharmaceutical formulation according to the invention preferably is characterized by at least one of the following features: (i) it is adapted to be administered by oral, parenteral (e.g., intramuscular, intraperitoneal, intravenous or subcutaneous injection, or implant), nasal, vaginal, rectal, sublingual, or topical routes of administration and can be formulated in dosage forms appropriate for each route of administration; and (ii) it is in unit dosage form, each unit dosage comprising an amount of a compound of formula (I) including GAL475, or a pharmaceutically acceptable salt, solvate, hydrate, enantiomer, stereoisomer, tautomer, or isotopic variant thereof, which is within the range of about 2 mg to about 1000 mg.

The formulations further can be characterized in that they can be administered alone or in combination with or in conjunction with other compounds which are known in the art to be useful for the treatment of metabolic syndrome or for the induction of weight loss.

In one aspect the compound of this invention is used to treat metabolic syndrome where the word “treat” as used herein means to alleviate or cure a disease, disorder or condition or to ease at least one symptom of the disease, disorder or condition.

In another aspect the compound of this invention is used to induce weight loss and decrease appetite.

In another aspect the compound of this invention is used to treat diabetes. In preferred embodiments, the disease or disorder is one suffered by humans and the compound of the invention are administered to humans.

The compound of the invention can be administered alone or in combination with other agents known to be beneficial in treating the disease, disorder or condition to be treated. As used herein, “in combination” means that GAL475 and the other agent can be co-administered, either in concomitant therapy or in a fixed physical combination, or they may be administered at separate times but so as to complement one another.

In the treatment or prevention of the foregoing conditions, broadly defined as metabolic syndrome, the compound can be administered alone or in combination with other compounds with therapeutic properties e.g., GLP-1 agonists, antidiabetic, insulin, anti-inflammatory, antioxidant, hypnotics, anxiolytics, antipsychotics, anorectic, melatonin agonists and antagonists, melatonin, benzodiazepines, Alpha-glucosidase inhibitors, Biguanides, Dopamine-2 agonist, DPP-4 inhibitors, Meglitinides, SGLT2 inhibitors, Sulfonylureas, Thiazolidinediones and the like, such as: Phentermine, Diethylpropion, orlistat, phentermine-topiramate, bupropion-naltrexone, liraglutide, setmelanotide, semaglutide, retatrutide, Tirzepatide, danuglipron, insulin, acarbose, miglitol, metformin and metformin combinations, Bromocriptine, alogliptin, linagliptin, saxagliptin, sitagliptin, dulaglutide, exenatide, lixisenatide, nateglinide, repaglinide, canagliflozin, dapagliflozin, empagliflozin, ertugliflozin, glimepiride, gliclazide, glyburide, rosiglitazone, pioglitazone zopiclone, and salts thereof, and combinations thereof.

Combinations of one or more of these known therapeutic agents with GAL475 will provide additional, complementary, and often synergistic effects to enhance the desirable properties of the known therapeutic agent.

GAL475, alone or in combination with one of the aforementioned known therapeutic agents further can be administered in combination with physical treatment methods, such as physical exercise, diet etc.

GAL475 can be formulated into pharmaceutical composition suitable for oral, parenteral (e.g., intramuscular, intraperitoneal, intravenous or subcutaneous injection, or implant) nasal, vaginal, rectal, sublingual or topical routes of administration. The compositions can comprise one or more pharmaceutically acceptable diluents, preservatives, solubilizers, emulsifiers, adjuvants, excipients and/or carriers.

Solid dosage forms for oral administration include capsules, tablets, pills, minitablets, pellets, powders and granules. In such solid dosage forms, the active compound is admixed with at least one inert pharmaceutically acceptable carrier such as sucrose, lactose, or starch. Such dosage forms can also comprise, as is normal practice, additional substances other than inert diluents, e.g., lubricating agents such as magnesium stearate. Illustrative of the adjuvants which may be incorporated in tablets, capsules and the like are the following: a binder such as gum tragacanth, acacia, corn starch or gelatin; an excipient such as microcrystalline cellulose; a disintegrating agent such as corn starch, pregelatinized starch, alginic acid and the like; a lubricant such as magnesium stearate; a sweetening agent such as sucrose, lactose or saccharin; a flavoring agent such as peppermint, oil of wintergreen or cherry. In the case of capsules, tablets and pills, the dosage forms may also comprise buffering agents. When the dosage unit form is a capsule, it may contain, in addition to materials of the above type, a liquid carrier such as a fatty oil. Various other materials can be present as coatings or to otherwise modify the physical form of the dosage unit. Tablets and pills can additionally be prepared with enteric coatings and tablets may be coated with shellac, sugar or both. Tablets may include but are not limited to matrix tablets, film-coated tablets, osmotic tablets, multi-layer tablets (for example, bi-layer, or tri-layer), traditional tablets, and the like.

Solid compositions of a similar type may be employed as fillers in soft and/or hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the provided composition(s) only in, or targeting, a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

In certain aspects, capsules may contain an excipient formulation containing one or more of hydroxypropyl methylcellulose (HPMC), gelatin, meglumine, and fish gelatin. In certain aspects, a capsule may contain a compound of formula (I) or a pharmaceutically acceptable salt, solvate, hydrate, enantiomer, stereoisomer, tautomer, or isotopic variant thereof, and in particular aspects. The capsule may optionally further contain one or more of lycopene, ellagic acid (polyphenol), curcumin, piperine, delphinidin, resveratrol, isothiocyanates such as sulforaphane, capsaicin, and piperlongumine.

When used in the form of microparticles or nanoparticles, the compounds of the claimed invention may achieve higher blood levels. The present invention includes microparticles and/or nanoparticles of the compounds of the present disclosure in tablet form or encapsulated in capsules.

Liquid dosage forms for oral administration include pharmaceutically acceptable emulsions, solutions, suspensions, Self-microemulsifying drug delivery system (SMEDDS), liposomes, syrups and elixirs containing inert diluents commonly used in the art, such as water. Besides such inert diluents, compositions can also include adjuvants, such as wetting agents, emulsifying and suspending agents, and sweetening, flavoring, and perfuming agents, oils, surfactants and co-surfactants. A syrup or elixir may contain the active compound, sucrose as a sweetening agent, methyl and propyl parabens as preservatives, a dye and a flavoring such as cherry or orange flavor.

Formulations in the form of an orally administered SMEDDS will increase the bioavailability and pharmacokinetics of GAL475 and/or a second agent. The formulation generally includes an emulsion including an oil or lipid material, a surfactant, and a hydrophilic co-surfactant. A poorly water-soluble drug or pharmaceutical is emulsified in the self-microemulsifying excipient formulation thereby increasing the in vivo bioavailability of the drug or pharmaceutical formulation. Additionally, poorly water-soluble drugs, can then be used in combination with GAL475.

SMEDDS pharmaceutical compositions of the invention may be formulated for oral administration of GAL475, e.g., emulsions, aqueous or oily suspensions, filled hard or soft capsules or syrups or prepared powders or granules, troches, tablets or lozenges. Pharmaceutically acceptable carriers or additives to the formulation include binders such as lactose, saccharose, sorbitol, mannitol, starch, amylopectin, cellulose or gelatin; Excipients such as dicalcium phosphate; Disintegrants such as corn starch or sweet potato starch; Glidants such as magnesium stearate, calcium stearate, sodium stearyl fumarate may also be included.

Preparations according to this invention for parenteral administration include sterile aqueous or non-aqueous solutions, suspensions, or emulsions. Sterile compositions for injection can be formulated according to conventional pharmaceutical practice by dissolving or suspending the active substance in a vehicle such as water for injection, a naturally occurring vegetable oil like sesame oil, coconut oil, peanut oil, cottonseed oil, etc., or a synthetic fatty vehicle like ethyl oleate or the like. Buffers, preservatives, antioxidants and the like may be incorporated as required. Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized by, for example, filtration through a bacteria-retaining filter, by incorporating sterilizing agents into the compositions, by irradiating the compositions, or by heating the compositions. They can also be manufactured in the form of sterile solid compositions that can be dissolved in sterile water, or some other sterile injectable medium immediately before use.

The dosage of active agent in compositions of this invention can vary, provided that a therapeutic amount is administered. Desirably the active agent is administered to a patient (human or animal) in need of such treatment in dosages that will provide optimal pharmaceutical efficacy. The selected dosage depends upon the nature and severity of the disease or disorder to be treated, desired therapeutic effect, the route of administration, and the duration of treatment. Dosage amount also can vary depending on the weight of the patient, and other factors. For example, the dose of GAL475 that reduces weight may be different than the dose required to alleviate diabetes. The dose will vary from patient to patient depending on the nature and severity of the disease, the patient's weight, special diets then being followed by the patient, concurrent medication, the bioavailability of the compound upon administration and other factors which those skilled in the art will recognize.

In the treatment of a condition in accordance with the present invention, an appropriate daily dosage level will generally be about 2-1000 mg. The daily dosage amount can be administered in single or multiple doses per day. Preferably, the dosage level will be about 100-500 mg.

Alternative treatment with the present invention can include bidaily, biweekly, weekly, bimonthly and monthly administrations.

Formulations of this invention can be in the form of immediate release, or can be in extended, controlled, release forms. Extended release formulations include delayed-, sustained-, pulsed- or controlled-release formulations. Suitable extended release formulations useful for purposes of the present invention include the types of formulations described in U.S. Pat. Nos. 6,106,864; 7,053,122; and 7,118,762, incorporated herein by reference. Details of other types of suitable release technologies, such as high energy dispersions and osmotic and coated particles can be found, for example, in Verma, R. and S. Garg, Pharmaceutical Technology On-Line, 25(2), 1-14 (2001), also incorporated herein by reference.

The period of time in which an extended release formulation releases the compound varies based upon the indication and the target therapeutic levels. For diabetes, for example, it is desirable to limit the pharmacological effects of the compound on glucose bioavailability to a few hours.

The present disclosure includes the following non-limiting aspects:
1. A method of:
   a) treating a subject suffering from a metabolic syndrome or disease;
   b) lowering body weight in a subject in need thereof,
   c) treating a subject having diabetes or at risk of developing diabetes;
   d) treating a subject having obesity or an overweight condition;
   e) treating a subject having a food intake disorder;
   f) treating a subject having hyperphagia;
   g) treating a subject having hyperlipidemia; or
   h) treating a subject having insulin resistance, comprising administering to the subject a composition comprising a compound of formula (I):

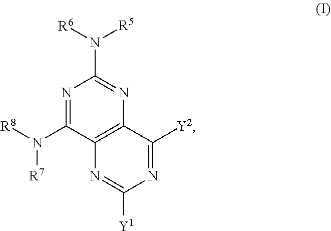

wherein in (I): one of the substituents selected from the group consisting of Y¹ and Y² is selected from the group consisting of —N(R¹¹)—L—C(R⁹)(R¹⁰)OH,

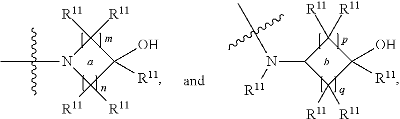

and the other substituent is —N(R¹)R²;
R¹, R⁵ and R⁷ are independently selected from the group consisting of hydrogen and optionally substituted C₁-C₃ alkyl;
R² is selected from the group consisting of alkyl, cycloalkyl, alkenyl, alkynyl, phenyl, phenylalkyl, aryl, arylalkyl, heteroarylalkyl and heteroaryl, wherein the alkyl, cycloalkyl, alkenyl, alkynyl, phenyl, phenylalkyl, aryl, arylalkyl, heteroarylalkyl or heteroaryl group is independently optionally substituted;
R⁶ and R⁸ are independently selected from the group consisting of alkyl, cycloalkyl, alkenyl, alkynyl, phenyl, phenylalkyl, aryl, arylalkyl, heteroarylalkyl and heteroaryl,
wherein the alkyl, cycloalkyl, alkenyl, alkynyl, phenyl, phenylalkyl, aryl, arylalkyl, heteroarylalkyl or heteroaryl group is independently optionally substituted;
R⁹ and R¹⁰ are independently selected from the group consisting of H and optionally substituted C₁-C₃-alkyl; or R⁹ and R¹⁰ combine with the carbon atom to which they are bound so as to form an optionally substituted C₃-C₆ cycloalkyl group;
each instance of R¹¹ is independently selected from the group consisting of H and optionally substituted C₁-C₃-alkyl; wherein a —C(R¹¹)₂—C(R¹¹)₂— group within ring b is optionally replaced by an optionally substituted 1,2-phenylene group that is fused with ring b;
each occurrence of independently optionally substituted C₁-C₃ alkylene;
m and n are independently selected from the group consisting of 1, 2, 3 and 4, such that 2≤(m+n)≤4;

$$2 \leq ( m + n ) \leq 4 ;$$

p and q are independently elected from the group consisting of 0, 1, 2, 3 and 4, such that

$$2 \leq ( p + q ) \leq 4 ;$$

with the proviso that the alkyl group is not substituted with a hydroxy group, or a pharmaceutically acceptable salt, solvate, hydrate, enantiomer, stereoisomer, tautomer, or isotopic variant thereof, and a pharmaceutically acceptable carrier or excipient.
2. A method of treating a subject suffering from a metabolic syndrome or disease comprising administering a composition comprising GAL475 (1-(2,6-Bis-methylamino-8-propylamino-pyrimido[5,4-d]pyrimidin-4-yl amino)-2-methyl-propan-2-ol), or a pharmaceutically acceptable salt, solvate, hydrate, enantiomer, stereoisomer, tautomer, or isotopic variant thereof, and a pharmaceutically acceptable carrier or excipient, to the subject.
3. A method of lowering body weight in a subject in need thereof, the method comprising administering a composition comprising GAL475 (1-(2,6-Bis-methylamino-8-propylamino-pyrimido[5,4-d]pyrimidin-4-yl amino)-2-methyl-propan-2-ol), or a pharmaceutically acceptable salt, solvate, hydrate, enantiomer, stereoisomer, tautomer, or isotopic variant thereof, and a pharmaceutically acceptable carrier or excipient, to the subject.
4. A method of treating a subject having diabetes or at risk of developing diabetes, the method comprising administering a composition comprising GAL475 (1-(2,6-Bis-methylamino-8-propylamino-pyrimido[5,4-d]pyrimidin-4-yl amino)-2-methyl-propan-2-ol), or a pharmaceutically acceptable salt, solvate, hydrate, enantiomer, stereoisomer, tautomer, or isotopic variant thereof, and a pharmaceutically acceptable carrier or excipient, to the subject.
5. A method of treating a subject having obesity or an overweight condition, the method comprising administering a composition comprising GAL475 (1-(2,6-Bis-methylamino-8-propylamino-pyrimido[5,4-d]pyrimidin-4-yl amino)-2-methyl-propan-2-ol), or a pharmaceutically acceptable salt, solvate, hydrate, enantiomer, stereoisomer, tautomer, or isotopic variant thereof, and a pharmaceutically acceptable carrier or excipient, to the subject.
6. A method of treating a subject having a food intake disorder, the method comprising administering a composition comprising GAL475 (1-(2,6-Bis-methylamino-8-propylamino-pyrimido[5,4-d]pyrimidin-4-yl amino)-2-methyl-propan-2-ol), or a pharmaceutically acceptable salt, solvate, hydrate, enantiomer, stereoisomer, tautomer, or isotopic variant thereof, and a pharmaceutically acceptable carrier or excipient, to the subject.
7. A method of treating a subject having hyperphagia, the method comprising administering a composition comprising GAL475 (1-(2,6-Bis-methylamino-8-propylamino-pyrimido[5,4-d]pyrimidin-4-yl amino)-2-methyl-propan-2-ol), or a pharmaceutically acceptable salt, solvate, hydrate, enantiomer, stereoisomer, tautomer, or isotopic variant thereof, and a pharmaceutically acceptable carrier or excipient, to the subject.
8. A method of treating a subject having hyperlipidemia, the method comprising administering a composition comprising GAL475 (1-(2,6-Bis-methylamino-8-propylamino-pyrimido[5,4-d]pyrimidin-4-yl amino)-2-methyl-propan-2-ol), or a pharmaceutically acceptable salt, solvate, hydrate, enantiomer, stereoisomer, tautomer, or isotopic variant thereof, and a pharmaceutically acceptable carrier or excipient, to the subject.
9. A method of treating a subject having insulin resistance, the method comprising administering a composition comprising GAL475 (1-(2,6-Bis-methylamino-8-propylamino-pyrimido[5,4-d]pyrimidin-4-yl amino)-2-methyl-propan-2-ol), or a pharmaceutically acceptable salt, solvate, hydrate, enantiomer, stereoisomer, tautomer, or isotopic variant thereof, and a pharmaceutically acceptable carrier or excipient, to the subject.
10. The method of any one of items 1-9, wherein the composition is a modified-release composition.
11. The method of item 10, wherein the modified release composition is an oral modified-release composition.
12. The method of any one of items 1-11, wherein the composition comprises an enteric coating.
13. The method of any one of items 1-12, wherein the composition comprises a gelatin coating.
14. The method of any one of items 1-13, wherein the composition comprises an excipient, wherein the excipient comprises a binder, disintegrant, diluent, buffer, lubricant, glidant, antioxidant, antimicrobial preservative, colorant, or flavorant.
15. The method of any one of items 1-14, wherein the compound is coated onto a base particle so as to form a core.
16. The method of item 15, wherein the core is coated with an enteric coating, thereby forming an enterically coated bead.
17. The method of any one of items 1-16, wherein the dosage form is an oral dosage form.
18. The method of item 17, wherein the oral dosage form is in the form of a capsule, a tablet, or a pharmaceutically acceptable solution.
19. The method of any one of items 1-18, wherein the composition comprises about 2 mg to about 5000 mg, about 5 mg to about 3000 mg, about 10 mg to about 2000 mg, about 20 mg to about 1500 mg, about 30 mg to about 1000 mg, about 40 mg to about 900 mg, about 50 mg to about 800 mg, about 100 mg to about 700 mg, about 150 mg to about 600 mg, about 200 mg to about 500 mg, about 250 mg to about 400 mg, or about 300 mg to about 350 mg of the compound.
20. The method of any one of items 1-19, wherein the composition is encapsulated in a capsule.
21. The method of item 20, wherein the capsule contains granules or powders of the compound, or granules or powder comprising a mixture of the compound with the pharmaceutically acceptable carrier or excipient.
22. The method of item 21, wherein the capsule is enterically coated but the granules or powders are not enterically coated.
23. The method of item 21, wherein at least a portion of the granules or powders are enterically coated.
24. The method of item 23, wherein the at least a portion of the granules or powders are coated with an enteric coating before encapsulation in the capsule.
25. The method of item 23 or item 24, comprising a first subportion of the granules or the powders coated with one enteric coating and at least a second subportion of the granules or the coated with a different enteric coating, wherein the first subportion is released in a different region of the intestine of the subject than the second subportion.
26. The method of any one of items 20-25, wherein the capsule is a liquid-filled capsule further comprising the composition and a pharmaceutically acceptable liquid mixed to form a liquid formulation.
27. The method of item 26, wherein the capsule is enterically coated, and wherein the liquid formulation in the capsule does not comprise an enteric coating.
28. The method of any one of items 1-27, further comprising administering at least one agent known for treating the underlying disease.
29. The method of any one of items 1-27, further comprising at least one additional agent selected from the group consisting of an antidiabetic agent, a weight loss agent, a metabolic syndrome treatment agent, and an anti-obesity agent.
30. The method of any one of items 28 and 29, comprising administering the composition or capsule separately from the at least one agent.
31. The method of any one of items 28 and 29, comprising co-administering the composition or capsule with at least one agent.
32. The method of any one of items 28 and 29, wherein the compound and the at least one additional agent are physically mixed in the composition.
33. The method of any one of items 28 and 29, wherein the compound and the at least one additional agent are configured in a physically-separated structure in the composition.
34. The method of any one of items 1-33, wherein the subject is a mammal or a bird.
35. The method of any one of items 1-33, wherein the subject is a human.
36. The method of any one of items 1-9, wherein the administering is by at least one route selected from the group consisting of nasal, inhalational, topical, oral, buccal, rectal, pleural, peritoneal, vaginal, intramuscular, subcutaneous, transdermal, epidural, intrathecal, and intravenous.
37. The method of any one of items 1-36, wherein the pharmaceutical acceptable salt comprises an acid addition salt selected from the group consisting of sulfuric, hydrochloric, hydrobromic, hydroiodic, nitric, carbonic, phosphoric, formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, pamoic, methanesulfonic, ethanesulfonic, benzenesulfonic, pantothenic, sulfanilic, stearic, alginic, trifluoromethanesulfonic, 2-hydroxyethanesulfonic, p-tolucncsul ionic, cyclohexylaminosulfonic, O-hydroxybutyric, salicylic, galactaric and galacturonic, or a combination thereof.
38. Use of GAL475 (1-(2,6-Bis-methylamino-8-propylamino-pyrimido[5,4-d]pyrimidin-4-yl amino)-2-methyl-propan-2-ol), and a pharmaceutically acceptable salt, solvate, hydrate, enantiomer, stereoisomer, tautomer, or isotopic variant thereof, in the manufacture of a medicament for inhibiting or treating a metabolic syndrome or disease in a subject in need thereof.
39. Use of GAL475 (1-(2,6-Bis-methylamino-8-propylamino-pyrimido[5,4-d]pyrimidin-4-yl amino)-2-methyl-propan-2-ol), and a pharmaceutically acceptable salt, solvate, hydrate, enantiomer, stereoisomer, tautomer, or isotopic variant thereof, in the manufacture of a medicament for inducing weight loss in a subject in need thereof.
40. Use of GAL475 (1-(2,6-Bis-methylamino-8-propylamino-pyrimido[5,4-d]pyrimidin-4-yl amino)-2-methyl-propan-2-ol), and a pharmaceutically acceptable salt, solvate, hydrate, enantiomer, stereoisomer, tautomer, or isotopic variant thereof, in the manufacture of a medicament for treating or preventing diabetes in a subject in need thereof.
41. Use of GAL475 (1-(2,6-Bis-methylamino-8-propylamino-pyrimido[5,4-d]pyrimidin-4-yl amino)-2-methyl-propan-2-ol), and a pharmaceutically acceptable salt, solvate, hydrate, enantiomer, stereoisomer, tautomer, or isotopic variant thereof, in the manufacture of a medicament for treating a food intake disorder in a subject in need thereof.
42. Use of GAL475 (1-(2,6-Bis-methylamino-8-propylamino-pyrimido[5,4-d]pyrimidin-4-yl amino)-2-methyl-propan-2-ol), and a pharmaceutically acceptable salt, solvate, hydrate, enantiomer, stereoisomer, tautomer, or isotopic variant thereof, in the manufacture of a medicament for treating hyperphagia in a subject in need thereof.
43. Use of GAL475 (1-(2,6-Bis-methylamino-8-propylamino-pyrimido[5,4-d]pyrimidin-4-yl amino)-2-methyl-propan-2-ol), and a pharmaceutically acceptable salt, solvate, hydrate, enantiomer, stereoisomer, tautomer, or isotopic variant thereof, in the manufacture of a medicament for treating hyperlipidemia in a subject in need thereof.
44. Use of GAL475 (1-(2,6-Bis-methylamino-8-propylamino-pyrimido[5,4-d]pyrimidin-4-yl amino)-2-methyl-propan-2-ol), and a pharmaceutically acceptable salt, solvate, hydrate, enantiomer, stereoisomer, tautomer, or isotopic variant thereof, in the manufacture of a medicament for treating insulin resistance in a subject in need thereof.
45. Use of a compound of formula (I):

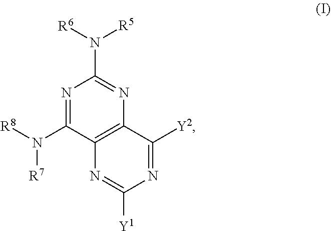

wherein in (I): one of the substituents selected from the group consisting of Y¹ and Y² is selected from the group consisting of —N(R¹¹)—L—C(R⁹)(R¹⁰)OH,

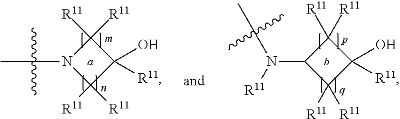

and the other substituent is —N(R¹)R²;
R¹, R⁸ and R⁷ are independently selected from the group consisting of hydrogen and optionally substituted C₁-C₃ alkyl;
R² is selected from the group consisting of alkyl, cycloalkyl, alkenyl, alkynyl, phenyl, phenylalkyl, aryl, arylalkyl, heteroarylalkyl and heteroaryl, wherein the alkyl, cycloalkyl, alkenyl, alkynyl, phenyl, phenylalkyl, aryl, arylalkyl, heteroarylalkyl or heteroaryl group is independently optionally substituted;
R⁶ and R⁸ are independently selected from the group consisting of alkyl, cycloalkyl, alkenyl, alkynyl, phenyl, phenylalkyl, aryl, arylalkyl, heteroarylalkyl and heteroaryl,
wherein the alkyl, cycloalkyl, alkenyl, alkynyl, phenyl, phenylalkyl, aryl, arylalkyl, heteroarylalkyl or heteroaryl group is independently optionally substituted;
R⁹ and R¹⁰ are independently selected from the group consisting of H and optionally substituted C₁-C₃-alkyl; or R⁹ and R¹⁰ combine with the carbon atom to which they are bound so as to form an optionally substituted C₃-C₆ cycloalkyl group;
each instance of R¹¹ is independently selected from the group consisting of H and optionally substituted C₁-C₃-alkyl; wherein a —C(R^{D})₂—C(R¹¹)₂— group within ring b is optionally replaced by an optionally substituted 1,2-phenylene group that is fused with ring b;
each occurrence of independently optionally substituted C₁-C₃ alkylene;
m and n are independently selected from the group consisting of 1, 2, 3 and 4, such that 2≤(m+n)≤4;

$$2 \leq ( m + n ) \leq 4 ;$$

p and q are independently elected from the group consisting of 0, 1, 2, 3 and 4, such that

$$2 \leq ( p + q ) \leq 4 ;$$

with the proviso that the alkyl group is not substituted with a hydroxy group, or a pharmaceutically acceptable salt, solvate, hydrate, enantiomer, stereoisomer, tautomer, or isotopic variant thereof, in the manufacture of a medicament for:
a) treating a subject suffering from a metabolic syndrome or disease;
b) lowering body weight in a subject in need thereof,
c) treating a subject having diabetes or at risk of developing diabetes;
d) treating a subject having obesity or an overweight condition;
e) treating a subject having a food intake disorder;
f) treating a subject having hyperphagia;
g) treating a subject having hyperlipidemia; or
h) treating a subject having insulin resistance.

The invention is illustrated by the following Examples. The following examples are understood to be illustrative only and are not intended to limit the scope of the present invention in any way.

### EXAMPLES

The invention is now described with reference to the following Examples. These Examples are provided for the purpose of illustration only, and the invention is not limited to these Examples, but rather encompasses all variations that are evident as a result of the teachings provided herein.

### Example 1: GAL475 Enhanced the Binding of Neurotensin to the NTSR1 Receptor

GAL475 (30 micromolar) was tested in multiple receptor binding assays. The binding was calculated as a % inhibition of the binding of a radioactively labelled ligand specific for each target.

Inhibition higher than 50% of a certain receptor radioligand in the presence of 30 micromolar GAL475 was observed with 5-HT2B(h) (agonist radioligand; 75%), 5-HT5a(h) (agonist radioligand; 50.6%), 5-HT7 (h) (agonist radioligand; 79%), al adrenoreceptor (non-selective) (antagonist radioligand; 67.1%) and Na+channel (site 2) (antagonist radioligand; 75.1%). However, the potency of GAL 475 at these receptors was lower than 1 micromolar and therefore not considered to be pharmacologically interesting. An unexpected finding was however that at 30 micromolar, GAL475 enhanced rather than inhibited NTS1 (h) (agonist radioligand) by 32% suggesting it to be a positive allosteric modulator (PAM). GAL475 was subsequently tested for PAM activity at the human NTS1 (FAST-0330I) and NTS2 (FAST-0331I) receptors, at 100-50,000 nanomolar concentrations. Agonist activity of GAL475 is expressed as a percentage of the activity of the reference agonist (Neurotensin) at its EC100 (maximal response) concentration. Antagonist activity of GAL475 is expressed as a percentage of the inhibition of reference agonist activity at its EC80 (80% inhibition) concentration. The results shown in Tables 1-3. The assays were conducted with recombinant cell lines. Receptor accession numbers, cellular background and reference compounds are shown in Table 1.

TABLE 1

| | Accession | | Reference |
|---|---|---|---|
| Receptor | Number | Cell line | agonist |
| NTS1 | NP_002522.2 | CHO-K1-mt aequorin | Neurotensin |
| NTS2 | NP_036476.1 | CHO-K1-mt aequorin | SR 48692 |

GAL475 (100-50,000 nM) was tested for positive allosteric modulator (PAM) activity at the human NTS1 (FAST-0330I) and NTS2 (FAST-0331I) receptors (Tables 2 and 3). The results indicated that GAL475 was able to bind and activate the NTSR1 but not the NTSR2 receptor with 50% effect at 355 nM.

TABLE 2
allosteric binding of GAL475 to NTSR1

| Compound | % Activation Average at | EC50 | Hill |
|---|---|---|---|
| ID | maximal concentration | (nM) | Coefficient |
| GAL-475 | 26.48 | 355 | 11.62 |

TABLE 3
affinity of GAL475 to NTSR2

| Compound | % Activation Average at | EC50 |
|---|---|---|
| ID | maximal concentration | (nM) |
| GAL-475 | 0.21 | — |

### Example 2: Effect of 28 Days Repeated GAL475*2HCL Oral Dose with 14-Days Recovery on Body Weight Gain in Rats

One hundred and forty-two (71/sex) rats were randomly assigned to 4 groups and given vehicle [2% (w/v) CMC-Na, 1% (v/v) Tween 80 in 0.1 M phosphate buffer at pH 7.4±0.2] or Gal-475*2HCl at 20, 40 or 80 mg/kg/day once daily for 28 days by the oral gavage route.

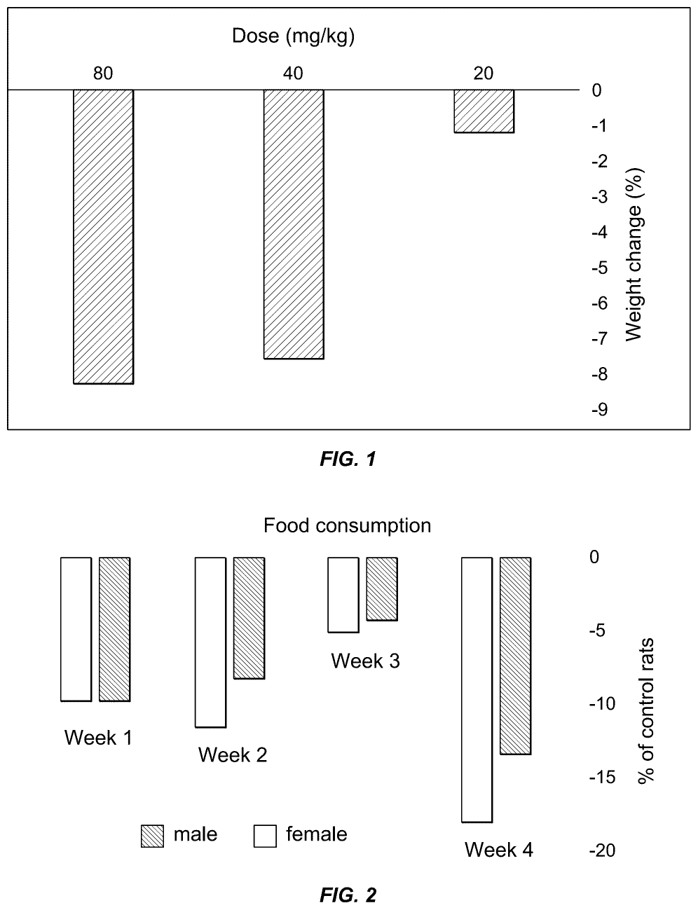

At the end of dosing phase, significantly lower group mean body weights were observed for animals given>40 mg/kg/day when compared to the controls (−7.35% and −4.70% for males and females respectively) (FIG. 1). However when the Day −1 data was compared to Day 28, the overall gain for those animals that had received 80 mg/kg/day was significantly reduced when compared to that of the controls (71% and 79% for males and females respectively). This reduction in group mean body weight was due to lower body weight gain which correlated and was attributed to the lower food intake. During a 2 weeks recovery phase (when Gal-475 was not administered) comparable or greater body weight gain was observed in animals at 80 mg/kg/day when compared to controls over the same period, indicating reversibility of GAL475 effect on body weight.

During dosing phase, when compared with the control group means, significant reduced group mean food consumption was noted in both sexes at >40 mg/kg/day, which contributed to the lower body weight gain (FIG. 2). However, as seen in the body weight the food intake reversed at the end of recovery phase.

GAL475 when administered by oral gavage once daily for 28 days to the rat at doses of 20, 40, or 80 mg/kg/day was well tolerated. There were no test article-related adverse effects in clinical signs, food consumption, clinical pathology, or pathology.

### Example 3: Effects of GAL475*2HCL: 28 Day Repeated Oral Dose with 14-Day Recovery on Body Weight Gain in Beagle Dogs

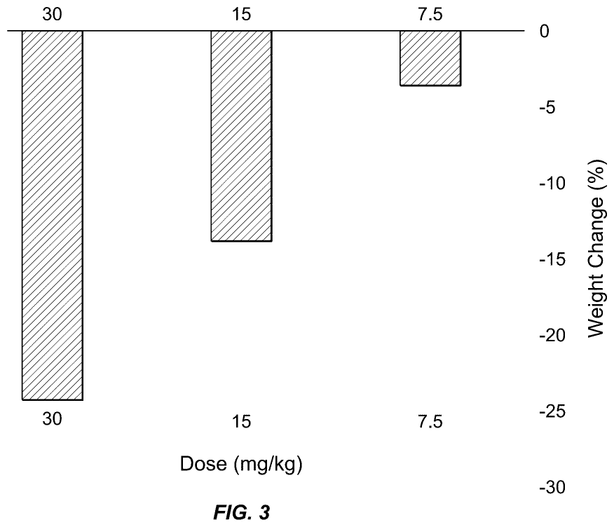

Thirty-two (16/sex) dogs were randomly assigned to 4 groups and orally administered vehicle (2% (w/v) CMC-Na, 1% (v/v) Tween 80 in 0.1 M phosphate buffer at pH 7.4) or Gal-475*2HCl at 7.5, 15, or 30 mg/kg/day once daily for up to 28 days. Decrease in body weight gain (up to 22.7% for males and 29.9% for females) (FIG. 3) and decrease in food consumption (up to 81% for males and 98% for females) was observed on all treated males and females (>7.5 mg/kg/day) during the dosing phase. During the recovery phase, the body weight gain and food consumption were comparable with the control.

### Example 4: Acute Gastric Emptying Inhibition Effects of GAL475 in Rats

Naive male Sprague-Dawley rats (239-297 g) were used to evaluate the effect on gastric emptying of a single dose of 3 mg/kg GAL-475 IV.

Rats were fasted 16-18 hours prior to the start of the experiment and then were given a bolus dose of either vehicle (1% tween-80 2% CMC) or GAL475 (3 mg/kg, IV) via the lateral tail vein. 5 minutes after dosing, rats were gavaged with a charcoal slurry (5 ml/kg, 10% charcoal in 2.5% Arabic gum from acacia tree) and then returned to their cages. 20 minutes after receiving the slurry, rats were euthanized using CO2. The stomachs were excised, weighed, emptied, washed and reweighed. Gastric content was calculated as the difference between full and empty weights. Increased gastric content was interpreted as a decrease in gastric emptying food retention).

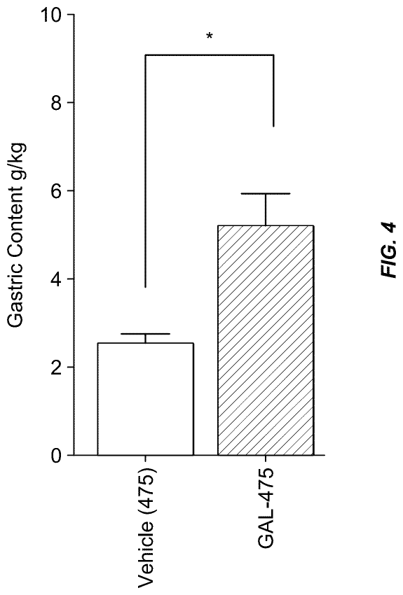

The effects of GAL475 and vehicle (25% DMA: 45% PEG300: 40% D5W) on gastric emptying in rats are presented (FIG. 4). GAL-475 significantly decreased gastric emptying (p<0.05) when compared to vehicle. Gastric content retention is higher by up to 105% compared to vehicle controls for up to 25 minutes after GAL-475 administration (3 mg/kg IV).

### Example 5: Weight Loss in Human Patients Treated with a Single Oral Dose of GAL475

The effects of an oral dose of GAL-475 (capsules of 450 mg) in 12 healthy male volunteers was tested in a Phase 1, randomized, placebo-controlled, double-blind, first in man single escalating dose study.

The study drug (GAL475) at 50, 150, 450 mg dosages, or placebo were administered on the morning of Day 1. Subjects fasted for 8 hours prior to the intake of study drug or placebo and for at least 4 hours following dosing.

The subject's body weight was measured on day −1 and at the follow up visit (day 7) using a validated balance. Body weight was recorded with 1 decimal. The treatment was well tolerated except for mild headaches in 2 of the 6 subjects taking 50 mg GAL475, 0 of the 6 subjects taking 150 mg GAL475, 5 of the 6 subjects taking 450 mg GAL475, and 1 of the 6 taking placebo on the day of capsule intake.

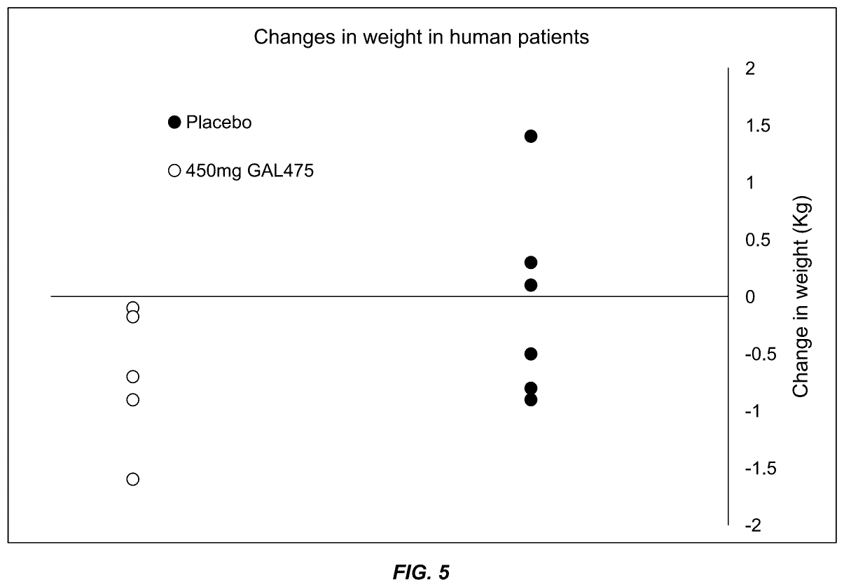

As shown in FIG. 5, analysis of body weight in subjects treated with 450 mg GAL475 revealed a consistent decrease in weight (average −0.6 kg, range −0.1 to −1.6 kg) in all subjects taking GAL-475, but not in those who were given placebo (Average −0.07 kg, range+1.4 kg to −0.8 kg). The weight decrease was observed a week after taking a single capsule only in subjects who took 450 mg GAL-475 and not placebo.

The effect of 450 mg GAL475 on weight loss was statistically significant (P=0.006) when compared to all other groups.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

All publications, patent applications, patents, and other references mentioned herein are incorporated by reference in their entirety. In case of conflict, the present specification, including definitions, will control. In addition section headings, the materials, methods, and examples are illustrative only and not intended to be limiting.

1. A method of:
a) treating a subject suffering from a metabolic syndrome or disease;
b) lowering body weight in a subject in need thereof,
c) treating a subject having diabetes or at risk of developing diabetes;
d) treating a subject having obesity or an overweight condition;
e) treating a subject having a food intake disorder;
f) treating a subject having hyperphagia;
g) treating a subject having hyperlipidemia; or
h) treating a subject having insulin resistance, comprising administering to the subject a composition comprising a compound of formula (I): 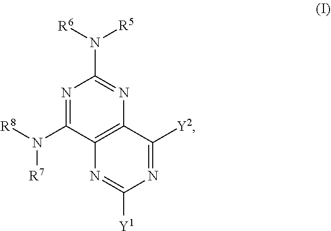
one of the substituents selected from the group consisting of Y¹ and Y² is selected from the group consisting of —N(R¹¹)—L—C(R⁹)(R¹⁰)OH, 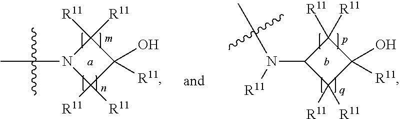
and the other substituent is —N(R¹)R²;
R¹, R⁸ and R⁷ are independently selected from the group consisting of hydrogen and optionally substituted C₁-C₃ alkyl;
R² is selected from the group consisting of alkyl, cycloalkyl, alkenyl, alkynyl, phenyl, phenylalkyl, aryl, arylalkyl, heteroarylalkyl and heteroaryl, wherein the alkyl, cycloalkyl, alkenyl, alkynyl, phenyl, phenylalkyl, aryl, arylalkyl, heteroarylalkyl or heteroaryl group is independently optionally substituted;
R⁶ and R⁸ are independently selected from the group consisting of alkyl, cycloalkyl, alkenyl, alkynyl, phenyl, phenylalkyl, aryl, arylalkyl, heteroarylalkyl and heteroaryl,
wherein the alkyl, cycloalkyl, alkenyl, alkynyl, phenyl, phenylalkyl, aryl, arylalkyl, heteroarylalkyl or heteroaryl group is independently optionally substituted;
R⁹ and R¹⁰ are independently selected from the group consisting of H and optionally substituted C₁-C₃-alkyl; or R⁹ and R¹⁰ combine with the carbon atom to which they are bound so as to form an optionally substituted C₃-C₆ cycloalkyl group;
each instance of R¹¹ is independently selected from the group consisting of H and optionally substituted C₁-C₃-alkyl; wherein a —C(R¹¹)₂—C(R¹¹)₂— group within ring b is optionally replaced by an optionally substituted 1,2-phenylene group that is fused with ring b;
each occurrence of independently optionally substituted C₁-C₃ alkylene;
m and n are independently selected from the group consisting of 1, 2, 3 and 4, such that
$$2 \leq ( m + n ) \leq 4 ;$$
p and q are independently elected from the group consisting of 0, 1, 2, 3 and 4, such that
$$2 \leq ( p + q ) \leq 4 ;$$
with the proviso that the alkyl group is not substituted with a hydroxy group, or a pharmaceutically acceptable salt, solvate, hydrate, enantiomer, stereoisomer, tautomer, or isotopic variant thereof, and a pharmaceutically acceptable carrier or excipient.

2. The method of claim 1, wherein the subject is suffering from a metabolic syndrome or disease and the method comprises administering a composition comprising GAL475 (1-(2,6-Bis-methylamino-8-propylamino-pyrimido[5,4-d]pyrimidin-4-yl amino)-2-methyl-propan-2-01), or a pharmaceutically acceptable salt, solvate, hydrate, enantiomer, stereoisomer, tautomer, or isotopic variant thereof, and a pharmaceutically acceptable carrier or excipient, to the subject.

3. The method of claim 1, wherein the subject has diabetes or is at risk of developing diabetes or has insulin resistance and the method comprises administering a composition comprising GAL475 (1-(2,6-Bis-methylamino-8-propylamino-pyrimido[5,4-d]pyrimidin-4-yl amino)-2-methyl-propan-2-ol), or a pharmaceutically acceptable salt, solvate, hydrate, enantiomer, stereoisomer, tautomer, or isotopic variant thereof, and a pharmaceutically acceptable carrier or excipient, to the subject.

4. The method of claim 1, wherein the subject has obesity or an overweight condition and the method comprises administering a composition comprising GAL475 (1-(2,6-Bis-methylamino-8-propylamino-pyrimido[5,4-d]pyrimidin-4-yl amino)-2-methyl-propan-2-ol), or a pharmaceutically acceptable salt, solvate, hydrate, enantiomer, stereoisomer, tautomer, or isotopic variant thereof, and a pharmaceutically acceptable carrier or excipient, to the subject.

5. The method of claim 1, wherein the subject has a food intake disorder and the method comprises administering a composition comprising GAL475 (1-(2,6-Bis-methylamino-8-propylamino-pyrimido[5,4-d]pyrimidin-4-yl amino)-2-methyl-propan-2-ol), or a pharmaceutically acceptable salt, solvate, hydrate, enantiomer, stereoisomer, tautomer, or isotopic variant thereof, and a pharmaceutically acceptable carrier or excipient, to the subject.

6. The method of claim 1, wherein the subject has hyperphagia and the method comprises administering a composition comprising GAL475 (1-(2,6-Bis-methylamino-8-propylamino-pyrimido[5,4-d]pyrimidin-4-yl amino)-2-methyl-propan-2-ol), or a pharmaceutically acceptable salt, solvate, hydrate, enantiomer, stereoisomer, tautomer, or isotopic variant thereof, and a pharmaceutically acceptable carrier or excipient, to the subject.

7. The method of claim 1, wherein the subject has hyperlipidemia and the method comprises administering a composition comprising GAL475 (1-(2,6-Bis-methylamino-8-propylamino-pyrimido[5,4-d]pyrimidin-4-yl amino)-2-methyl-propan-2-ol), or a pharmaceutically acceptable salt, solvate, hydrate, enantiomer, stereoisomer, tautomer, or isotopic variant thereof, and a pharmaceutically acceptable carrier or excipient, to the subject.

8. The method of claim 1, wherein the composition is a modified-release composition.

9. The method of claim 8, wherein the modified release composition is an oral modified-release composition.

10. The method of claim 1, wherein the composition comprises an enteric coating.

11. The method of claim 1, wherein the composition comprises a gelatin coating.

12. The method of claim 1, wherein the compound is coated onto a base particle so as to form a core.

13. The method of claim 12, wherein the core is coated with an enteric coating, thereby forming an enterically coated bead.

14. The method of claim 1, wherein the composition is an oral dosage form.

15. The method of claim 14, wherein the oral dosage form is in the form of a capsule, a tablet, or a pharmaceutically acceptable solution.

16. The method of claim 1, wherein the composition comprises about 2 mg to about 5000 mg, about 5 mg to about 3000 mg, about 10 mg to about 2000 mg, about 20 mg to about 1500 mg, about 30 mg to about 1000 mg, about 40 mg to about 900 mg, about 50 mg to about 800 mg, about 100 mg to about 700 mg, about 150 mg to about 600 mg, about 200 mg to about 500 mg, about 250 mg to about 400 mg, or about 300 mg to about 350 mg of the compound.

17. The method of claim 1, wherein the composition is encapsulated in a capsule.

18. The method of claim 17, wherein the capsule contains granules or powders of the compound, or granules or powder comprising a mixture of the compound with the pharmaceutically acceptable carrier or excipient.

19. The method of claim 17, wherein the capsule is enterically coated but the granules or powders are not enterically coated.

20. The method of claim 19, wherein at least a portion of the granules or powders are enterically coated.

21. The method of claim 19, wherein the at least a portion of the granules or powders are coated with an enteric coating before encapsulation in the capsule.

22. The method of claim 19, comprising a first subportion of the granules or the powders coated with one enteric coating and at least a second subportion of the granules or the coated with a different enteric coating, wherein the first subportion is released in a different region of the intestine of the subject than the second subportion.

23. The method of claim 17, wherein the capsule is a liquid-filled capsule further comprising the composition and a pharmaceutically acceptable liquid mixed to form a liquid formulation.

24. The method of claim 23, wherein the capsule is enterically coated, and wherein the liquid formulation in the capsule does not comprise an enteric coating.
